SEQUENCE LISTING (81 sequences)

SEQ ID NO: 1 - protein, 5 aa, synthetic construct
NYAMS

SEQ ID NO: 2 - protein, 9 aa, synthetic construct
SISPGDSSI

SEQ ID NO: 3 - protein, 8 aa, synthetic construct
KLRGAFDY

SEQ ID NO: 4 - protein, 13 aa, synthetic construct
SGSSSNIGSNAVS

SEQ ID NO: 5 - protein, 4 aa, synthetic construct
ANSH

SEQ ID NO: 6 - protein, 9 aa, synthetic construct
ASWDASLNA

SEQ ID NO: 7 - protein, 5 aa, synthetic construct
GYDMS

SEQ ID NO: 8 - protein, 9 aa, synthetic construct
GISYSGGST

SEQ ID NO: 9 - protein, 13 aa, synthetic construct
KDVLRGPPYDFDY

SEQ ID NO: 10 - protein, 13 aa, synthetic construct
SGSSSNIGSNTVS

SEQ ID NO: 11 - protein, 4 aa, synthetic construct
ADNN

SEQ ID NO: 12 - protein, 9 aa, synthetic construct
GTWDYSLSG

SEQ ID NO: 13 - protein, 5 aa, synthetic construct
NYYMS

SEQ ID NO: 14 - protein, 9 aa, synthetic construct
LISYGGSNT

SEQ ID NO: 15 - protein, 19 aa, synthetic construct
KYSRGQRGTTHYSDDGMDV

SEQ ID NO: 16 - protein, 13 aa, synthetic construct
SGSSSNIGNNHVS

SEQ ID NO: 17 - protein, 4 aa, synthetic construct
DDNQ

SEQ ID NO: 18 - protein, 9 aa, synthetic construct
GSWDASLNG

SEQ ID NO: 19 - protein, 5 aa, synthetic construct
GYSMS

SEQ ID NO: 20 - protein, 9 aa, synthetic construct
SISYGGSNK

SEQ ID NO: 21 - protein, 19 aa, synthetic construct
KPVGSVSQGQPTTANGMDV

SEQ ID NO: 22 - protein, 13 aa, synthetic construct
SGSSSNIGSNAVS

SEQ ID NO: 23 - protein, 4 aa, synthetic construct
SDSN

SEQ ID NO: 24 - protein, 9 aa, synthetic construct
GSWDDSLSG

SEQ ID NO: 25 - protein, 5 aa, synthetic construct
NYSMS

SEQ ID NO: 26 - protein, 9 aa, synthetic construct
LIYHGDGSK

SEQ ID NO: 27 - protein, 19 aa, synthetic construct
KGGLLPPWHPHYSANGMDV

SEQ ID NO: 28 - protein, 13 aa, synthetic construct
TGSSSNIGSNYVS

SEQ ID NO: 29 - protein, 4 aa, synthetic construct
SDSN

SEQ ID NO: 30 - protein, 9 aa, synthetic construct
GTWDSSLSG

SEQ ID NO: 31 - protein, 5 aa, synthetic construct
GYAMS

SEQ ID NO: 32 - protein, 9 aa, synthetic construct
AISHDDSSI

SEQ ID NO: 33 - protein, 19 aa, synthetic construct
KPRLPLHLYSLYYSDGMDV

SEQ ID NO: 34 - protein, 13 aa, synthetic construct
SGSSSNIGNNYVS

SEQ ID NO: 35 - protein, 4 aa, synthetic construct
ADSK

SEQ ID NO: 36 - protein, 9 aa, synthetic construct
ASWDYSLSG

SEQ ID NO: 37 - protein, 5 aa, synthetic construct
NYAMS

SEQ ID NO: 38 - protein, 9 aa, synthetic construct
SISPGDSSI

SEQ ID NO: 39 - protein, 8 aa, synthetic construct
KRVPAFDY

SEQ ID NO: 40 - protein, 13 aa, synthetic construct
SGSSSNIGSNYVS

SEQ ID NO: 41 - protein, 4 aa, synthetic construct
SNSH

SEQ ID NO: 42 - protein, 9 aa, synthetic construct
DTWDSSLSG

SEQ ID NO: 43 - protein, 5 aa, synthetic construct
NYDMS

SEQ ID NO: 44 - protein, 9 aa, synthetic construct
SIYPNGSSK

SEQ ID NO: 45 - protein, 19 aa, synthetic construct
RGRIACMYYTCSYDNAMDV

SEQ ID NO: 46 - protein, 13 aa, synthetic construct
SGSSSNIGSNAVN

SEQ ID NO: 47 - protein, 4 aa, synthetic construct
ADSN

SEQ ID NO: 48 - protein, 9 aa, synthetic construct
GTWDYSLSG

SEQ ID NO: 49 - protein, 5 aa, synthetic construct
SYDMS

SEQ ID NO: 50 - protein, 9 aa, synthetic construct
GISYGGSNT

SEQ ID NO: 51 - protein, 19 aa, synthetic construct
KPSLSQRSTFLSSANGMDV

SEQ ID NO: 52 - protein, 13 aa, synthetic construct
SGSSSNIGNNTVY

SEQ ID NO: 53 - protein, 4 aa, synthetic construct
DDSN

SEQ ID NO: 54 - protein, 9 aa, synthetic construct
GTWDDSLSG

SEQ ID NO: 55 - protein, 116 aa, synthetic construct
EVQLLESGGGLVQPGGSLRLSCAASGFTFSNYAMSWVRQAPGKGLEWVSSISPGDSSIYYADSVKGRFAISRDNSKNTLYLQMNSLRAEDTAVYYCAKLRGAFDYWGQGTLVTVSS

SEQ ID NO: 56 - protein, 110 aa, synthetic construct
QSVPTQPPSASGTPGQRVTISCSGSSSNIGSNAVSWYQQLPGTAPKLLIYANSHRPSGVPDRFSGSKSGTSASLAISGLRSEDEADYYCASWDASLNAYVFGGGTKLTVL

SEQ ID NO: 57 - protein, 121 aa, synthetic construct
EVQLLESGGGLVQPGGSLRLSCAASGFTFSGYDMSWVRQAPGKGLEWVSGISYSGGSTYYADSVKGRFTISRDNSKNTLYLQMNSLRAEDTAVYYCAKDVLRGPPYDFDYWGQGTLVTASS

SEQ ID NO: 58 - protein, 110 aa, synthetic construct
QSVLTQPPSASGTPGQRVTISCSGSSSNIGSNTVSWYQQLPGTAPKLLIYADNNRPSGVPDRFSGSKSGTSASLAISGLRSEDEADYYCGTWDYSLSGYVFGGGTKLTVL

SEQ ID NO: 59 - protein, 127 aa, synthetic construct
EVQLLESGGGLVQPGGSLRLSCAASGFTFSNYYMSWVRQAPGKGLEWVSLISYGGSNTYYADSVKGRFTISRDNSKNTLYLQMNSLRAEDTAVYYCAKYSRGQRGTTHYSDDGMDVWGQGTLVTVSS

SEQ ID NO: 60 - protein, 110 aa, synthetic construct
QSVLTQPPSASGTPGQRVTISCSGSSSNIGNNHVSWYQQLPGTAPKLLIYDDNQRPSGVPDRFSGSKSGTSASLAISGLRSEDEADYYCGSWDASLNGYVFGGGTKLTVL

SEQ ID NO: 61 - protein, 127 aa, synthetic construct
EVQLLESGGGLVQPGGSLRLSCAASGFTFSGYSMSWVRQAPGKGLEWVSSISYGGSNKYYADSVKGRFTISRDNSKNTLYLQMNSLRAEDTAVYYCAKPVGSVSQGQPYYANGMDVWGQGTLVTVSS

SEQ ID NO: 62 - protein, 110 aa, synthetic construct
QSVLTQPPSASGTPGQRVTISCSGSSSNIGSNAVSWYQQLPGTAPKLLIYSDSNRPSGVPDRFSGSKSGTSASLAISGLRSEDEADYYCGSWDDSLSGYVFGGGTKLTVL

SEQ ID NO: 63 - protein, 127 aa, synthetic construct
EVQLLESGGGLVQPGGSLRLSCAASGFTFSNYSMSWVRQAPGKGLEWVSLIYHGDGSKYYADSVKGRFTISRDNSKNTLYLQMNSLRAEDTAVYYCAKGGLLPPWHPHYSANGMDVWGQGTLVTVSS

SEQ ID NO: 64 - protein, 110 aa, synthetic construct
QSVLTQPPSASGTPGQRVTISCTGSSSNIGSNYVSWYRQLPGTAPKLLIYSDSNRPSGVPDRFSGSKSGTSASLAISGLRSEDEADYYCGTWDSSLSGYVFGGGTKLTVL

SEQ ID NO: 65 - protein, 127 aa, synthetic construct
EVQLLESGGGLVQPGGSLRLSCAASGFTFSGYAMSWVRQAPGKGLEWVSAISHDDSSIYYADSVKGRFTISRDNSKNTLYLQMNSLRAEDTAVYYCAKPRLPLHLYSLYYSDGMDVWGQGTLVTVSS

SEQ ID NO: 66 - protein, 110 aa, synthetic construct
QSVLTQPPSASGTPGQRVTISCSGSSSNIGNNYVSWYQQLPGTAPKLLIYADSKRPSGVPDRFSGSKSGTSASLAISGLRSEDEADYYCASWDYSLSGYVFGGGTKLTVL

SEQ ID NO: 67 - protein, 116 aa, synthetic construct
EVQLLESGGGLVQPGGSLRLSCAASGFTFSNYAMSWVRQAPGKGLEWVSSISPGDSSIYYADSVKGRFTISRDNSKNTLYLQMNSLRAEDTAVYYCAKRVPAFDYWGQGTQVTVSS

SEQ ID NO: 68 - protein, 110 aa, synthetic construct
QSVLTQPPSASGTPGQRVTISCSGSSSNIGSNYVSWYQQLPGTAPKLLIYSNSHRPSGVPDRFSGSKSGTSASLAISGLRSEDEADYYCDTWDSSLSGYVFGGGTKLTVL

SEQ ID NO: 69 - protein, 127 aa, synthetic construct
EVQLLGSGGGLVQPGGSLRLSCAASGFTFSNYDMSWVRQAPGKGLEWVSSIYPNGSSKYYADSVKGRFTISRDNSKNTLYLQMNSLRAEDTAVYYCARGRIACMYYTCSYDNAMDVWGQGTLVTVSS

SEQ ID NO: 70 - protein, 110 aa, synthetic construct
QSVLTQPPSASGTPGQRVTISCSGSSSNIGSNAVNWYQQLPGTAPKLLIYADSNRPSGVPDRFSGSKSGTSASLAISGLRSEDEADYYCGTWDYSLSGYVFGGGTKLTVL

SEQ ID NO: 71 - protein, 127 aa, synthetic construct
EVQLLESGGGLVQPGGSLRLSCAASGFTFSSYDMSWVRQAPGKGLEWVSGISYGGSNTYYADSVKGRFTISRDNSKNTLYLQMNSLRAEDTAVYYCAKPSLSQRSTFLSSANGMDVWGQGTLVTVSS

SEQ ID NO: 72 - protein, 110 aa, synthetic construct
QSVLTQPPSASGTPGQRVTISCSGSSSNIGNNTVYWYQQLPGTAPKLLIYDDSNRPSGVPDRFSGSKSGTSASLAISGLRSEDEADYYCGTWDDSLSGYVFGGGTKLTVL

SEQ ID NO: 73 - protein, 701 aa, synthetic construct
MEFGLSWLFLVAILKGVQCEVQLLESGGGLVQPGGSLRLSCAASGFTFSNYAMSWVRQAPGKGLEWVSSISPGDSSIYYADSVKGRFAISRDNSKNTLYLQMNSLRAEDTAVYYCAKLRGAFDYWGQGTLVTVSSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVPTQPPSASGTPGQRVTISCSGSSSNIGSNAVSWYQQLPGTAPKLLIYANSHRPSGVPDRFSGSKSGTSASLAISGLRSEDEADYYCASWDASLNAYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 706 aa, synthetic construct
MEFGLSWLFLVAILKGVQCEVQLLESGGGLVQPGGSLRLSCAASGFTFSGYDMSWVRQAPGKGLEWVSGISYSGGSTYYADSVKGRFTISRDNSKNTLYLQMNSLRAEDTAVYYCAKDVLRGPPYDFDYWGQGTLVTASSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVLTQPPSASGTPGQRVTISCSGSSSNIGSNTVSWYQQLPGTAPKLLIYADNNRPSGVPDRFSGSKSGTSASLAISGLRSEDEADYYCGTWDYSLSGYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - protein, 712 aa, synthetic construct
MEFGLSWLFLVAILKGVQCEVQLLESGGGLVQPGGSLRLSCAASGFTFSNYYMSWVRQAPGKGLEWVSLISYGGSNTYYADSVKGRFTISRDNSKNTLYLQMNSLRAEDTAVYYCAKYSRGQRGTTHYSDDGMDVWGQGTLVTVSSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVLTQPPSASGTPGQRVTISCSGSSSNIGNNHVSWYQQLPGTAPKLLIYDDNQRPSGVPDRFSGSKSGTSASLAISGLRSEDEADYYCGSWDASLNGYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 76 - protein, 712 aa, synthetic construct
MEFGLSWLFLVAILKGVQCEVQLLESGGGLVQPGGSLRLSCAASGFTFSGYSMSWVRQAPGKGLEWVSSISYGGSNKYYADSVKGRFTISRDNSKNTLYLQMNSLRAEDTAVYYCAKPVGSVSQGQPYYANGMDVWGQGTLVTVSSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVLTQPPSASGTPGQRVTISCSGSSSNIGSNAVSWYQQLPGTAPKLLIYSDSNRPSGVPDRFSGSKSGTSASLAISGLRSEDEADYYCGSWDDSLSGYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - protein, 712 aa, synthetic construct
MEFGLSWLFLVAILKGVQCEVQLLESGGGLVQPGGSLRLSCAASGFTFSNYSMSWVRQAPGKGLEWVSLIYHGDGSKYYADSVKGRFTISRDNSKNTLYLQMNSLRAEDTAVYYCAKGGLLPPWHPHYSANGMDVWGQGTLVTVSSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVLTQPPSASGTPGQRVTISCTGSSSNIGSNYVSWYRQLPGTAPKLLIYSDSNRPSGVPDRFSGSKSGTSASLAISGLRSEDEADYYCGTWDSSLSGYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - protein, 712 aa, synthetic construct
MEFGLSWLFLVAILKGVQCEVQLLESGGGLVQPGGSLRLSCAASGFTFSGYAMSWVRQAPGKGLEWVSAISHDDSSIYYADSVKGRFTISRDNSKNTLYLQMNSLRAEDTAVYYCAKPRLPLHLYSLYYSDGMDVWGQGTLVTVSSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVLTQPPSASGTPGQRVTISCSGSSSNIGNNYVSWYQQLPGTAPKLLIYADSKRPSGVPDRFSGSKSGTSASLAISGLRSEDEADYYCASWDYSLSGYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 701 aa, synthetic construct
MEFGLSWLFLVAILKGVQCEVQLLESGGGLVQPGGSLRLSCAASGFTFSNYAMSWVRQAPGKGLEWVSSISPGDSSIYYADSVKGRFTISRDNSKNTLYLQMNSLRAEDTAVYYCAKRVPAFDYWGQGTQVTVSSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVLTQPPSASGTPGQRVTISCSGSSSNIGSNYVSWYQQLPGTAPKLLIYSNSHRPSGVPDRFSGSKSGTSASLAISGLRSEDEADYYCDTWDSSLSGYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 80 - protein, 712 aa, synthetic construct
MEFGLSWLFLVAILKGVQCEVQLLGSGGGLVQPGGSLRLSCAASGFTFSNYDMSWVRQAPGKGLEWVSSIYPNGSSKYYADSVKGRFTISRDNSKNTLYLQMNSLRAEDTAVYYCARGRIACMYYTCSYDNAMDVWGQGTLVTVSSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVLTQPPSASGTPGQRVTISCSGSSSNIGSNAVNWYQQLPGTAPKLLIYADSNRPSGVPDRFSGSKSGTSASLAISGLRSEDEADYYCGTWDYSLSGYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 81 - protein, 712 aa, synthetic construct
MEFGLSWLFLVAILKGVQCEVQLLESGGGLVQPGGSLRLSCAASGFTFSSYDMSWVRQAPGKGLEWVSGISYGGSNTYYADSVKGRFTISRDNSKNTLYLQMNSLRAEDTAVYYCAKPSLSQRSTFLSSANGMDVWGQGTLVTVSSASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMAGFPLLLTLLTHCAGSWAQSVLTQPPSASGTPGQRVTISCSGSSSNIGNNTVYWYQQLPGTAPKLLIYDDSNRPSGVPDRFSGSKSGTSASLAISGLRSEDEADYYCGTWDDSLSGYVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2026/0217862 A1
(43) Pub. Date: Jul. 30, 2026

(54) MONOCLONAL ANTIBODY SPECIFICALLY BINDING TO CATHEPSIN Z, AND USE THEREOF

(71) Applicants: NBIOS, INC., Gangneung-si, Gangwon-do (KR); KOOKMIN UNIVERSITY INDUSTRY ACADEMY COOPERATION FOUNDATION, Seoul (KR)

(72) Inventors: Dae-Hee LEE, Yongin-si (KR); Tae Hyun KANG, Seoul (KR); Han Gil KIM, Seoul (KR)

(73) Assignees: NBIOS, INC., Gangneung-si, Gangwon-do (KR); KOOKMIN UNIVERSITY INDUSTRY ACADEMY COOPERATION FOUNDATION, Seoul (KR)

(21) Appl. No.: 19/140,606

(22) PCT Filed: Dec. 19, 2023
(86) PCT No.: PCT/KR2023/020963

§ 371 Date: Jun. 18, 2025

(30) Foreign Application Priority Data
Dec. 22, 2022 (KR) 10-2022-0182137
May 9, 2023 (KR) 10-2023-0059737

(51) Int. Cl. C07K 16/40; A61P 35/00; G01N 33/575

(52) U.S. Cl. CPC C07K 16/40; A61P 35/00; C12Y 304/18001; G01N 33/57557; G01N 2333/948

(57) ABSTRACT

The present disclosure relates to a monoclonal antibody specifically binding to cathepsin Z, and a use thereof. The monoclonal antibody specifically binding to cathepsin Z of the present disclosure, or an antigen-binding fragment thereof specifically binds to CtsZ, and thus can inhibit in vivo tumorigenesis of cancer in which CtsZ is overexpressed, particularly, brain cancer, more particularly, mesenchymal brain cancer stem cells. Therefore, the present disclosure can be usefully used for treatment of patients with cancer in which CtsZ is overexpressed, specifically patients with mesenchymal brain cancer, more specifically patients with glioblastoma. In addition, the monoclonal antibody of the present disclosure or an antigen-binding fragment thereof, which specifically binds to cathepsin Z, allows diagnosis of cancer in which CtsZ is overexpressed, specifically brain cancer, more specifically glioblastoma.

### TECHNICAL FIELD

The present disclosure relates to a monoclonal antibody specifically binding to cathepsin Z, and a use thereof.

### BACKGROUND ART

Cancer is one of the leading causes of death around the world. About 10 million new cases occur every year. It accounts for about 12% of global mortality, and is the third leading cause of death. Among various types of cancer, brain cancer occurs regardless of age, and is characterized by a higher frequency of occurrence in children than other cancers. Brain cancer is classified into primary brain tumor, which occurs in the brain tissue and the meninges surrounding the brain, and secondary brain tumor metastasized from cancers occurring in the skull or other parts of the body, and symptoms include local symptoms such as motor paralysis, paralysis of sensation, speech disorder, problems with vision, balance disorder, etc. and intracranial hypertension symptoms. Unlike the cancers occurring only in specific tissues, brain cancer includes various types of cancers, such as glioblastoma multiforme, malignant glioma, lymphangioma, germ cell tumor, metastatic tumor, etc. Among them, glioma, particularly glioblastoma, has a very poor prognosis because it is the most malignant and aggressive. It is a very fatal disease with median survival of less than about 1 year. Since the boundary between brain cells and tumor cells is not clear in patients with glioblastoma, it is known to be almost impossible to completely remove the cancer tissue by surgery.

Therefore, various researches have been conducted to develop a method for treating glioblastoma. However, since glioblastoma includes various genetic mutations, the median survival rate of patients with glioblastoma is very low even when combined therapy is used. Accordingly, the development of a new therapeutic agent is required.

For brain tumor, the drug administered for the purpose of treatment is difficult to be delivered to the desired part of the brain due to the presence of the blood-brain barrier. In addition, the development of therapeutic agents is not active due to the relative lack of the understanding of neurobiology. Furthermore, since glioblastoma is an aggressive variant of brain tumor, fatal results may occur within a few weeks unless it is treated early.

Meanwhile, cathepsin Z (hereinafter, referred to as ‘CtsZ’) is known to induce tumorigenesis and invasiveness in various tumors, particularly mesenchymal glioblastoma. For example, the cathepsin Z may be human cathepsin Z. The information of the human cathepsin Z is registered as Accession No. AAH42168.1, etc. in the NCBI (National Center for Biotechnology Information), and the information of a gene encoding the same is registered as NCBI Accession No. AAV38718.1, etc. It is known that CtsZ is associated with tumorigenesis and invasiveness and, particularly, it is overexpressed in brain cancers, specifically mesenchymal glioblastoma stem cells (GSCs).

Therefore, the inventors of the present disclosure have made consistent efforts to develop a monoclonal antibody that specifically binds to CtsZ and can effectively inhibit the biological activity of CtsZ. As a result, they have newly constructed a monoclonal antibody that specifically binds to human CtsZ from an antibody library, and have completed the present disclosure by identifying that the antibody effectively inhibits the biological activity of CtsZ, specifically the in vivo tumorigenesis of cancer in which CtsZ is overexpressed, specifically brain cancer, more specifically mesenchymal brain cancer stem cells.

### DISCLOSURE

### Technical Problem

The present disclosure is directed to providing a monoclonal antibody specifically binding to cathepsin Z, or an antigen-binding fragment thereof.

The present disclosure is also directed to providing a polynucleotide encoding the monoclonal antibody or an antigen-binding fragment thereof, an expression vector including the polynucleotide, and a transgenic organism in which the expression vector is introduced.

The present disclosure is also directed to providing a pharmaceutical composition for preventing or treating cancer, which contains the monoclonal antibody or an antigen-binding fragment thereof, a polynucleotide encoding the monoclonal antibody or an antigen-binding fragment thereof, or an expression vector including the polynucleotide.

The present disclosure is also directed to providing a method for preventing or treating cancer, which includes a step of administering the monoclonal antibody or an antigen-binding fragment thereof, a polynucleotide encoding the monoclonal antibody or an antigen-binding fragment thereof, or an expression vector including the polynucleotide to a subject in need thereof.

The present disclosure is also directed to providing a composition for diagnosing cancer, which contains the monoclonal antibody or an antigen-binding fragment thereof, a polynucleotide encoding the monoclonal antibody or an antigen-binding fragment thereof, or an expression vector including the polynucleotide.

The present disclosure is also directed to providing a kit for diagnosing cancer, which contains the monoclonal antibody or an antigen-binding fragment thereof, a polynucleotide encoding the monoclonal antibody or an antigen-binding fragment thereof, or an expression vector including the polynucleotide.

The present disclosure is also directed to providing a method for providing information necessary for diagnosis of cancer, which includes: (a) a step of contacting the monoclonal antibody or an antigen-binding fragment thereof with a biological sample isolated from a subject suspected of cancer; (b) a step of measuring the expression level of the cathepsin Z protein bound to the monoclonal antibody or the antigen-binding fragment thereof in the biological sample through formation of an antigen-antibody complex; and (c) a step of diagnosing as cancer if the expression level of the cathepsin Z protein measured in the step (b) is higher as compared to a control group.

### Technical Solution

The present disclosure relates to a monoclonal antibody including any heavy chain variable region and light chain variable region selected from a group consisting of 1) to 9) and specifically binding to cathepsin Z, or an antigen-binding fragment thereof:
1) a heavy chain variable region including a CDR1 region represented by SEQ ID NO: 1, CDR2 region represented by SEQ ID NO: 2 and a CDR3 region represented by SEQ ID NO: 3, and a light chain variable region including CDR1 region represented by SEQ ID NO: 4, CDR2 region represented by SEQ ID NO: 5 and a CDR3 region represented by SEQ ID NO: 6;
2) a heavy chain variable region including a CDR1 region represented by SEQ ID NO: 7, a CDR2 region represented by SEQ ID NO: 8 and a CDR3 region represented by SEQ ID NO: 9, and a light chain variable region including a CDR1 region represented by SEQ ID NO: 10, a CDR2 region represented by SEQ ID NO: 11 and a CDR3 region represented by SEQ ID NO: 12;
3) a heavy chain variable region including a CDR1 region represented by SEQ ID NO: 13, a CDR2 region represented by SEQ ID NO: 14 and a CDR3 region represented by SEQ ID NO: 15, and a light chain variable region including a CDR1 region represented by SEQ ID NO: 16, a CDR2 region represented by SEQ ID NO: 17 and a CDR3 region represented by SEQ ID NO: 18;
4) a heavy chain variable region including a CDR1 region represented by SEQ ID NO: 19, a CDR2 region represented by SEQ ID NO: 20 and a CDR3 region represented by SEQ ID NO: 21, and a light chain variable region including a CDR1 region represented by SEQ ID NO: 22, a CDR2 region represented by SEQ ID NO: 23 and a CDR3 region represented by SEQ ID NO: 24;
5) a heavy chain variable region including a CDR1 region represented by SEQ ID NO: 25, a CDR2 region represented by SEQ ID NO: 26 and a CDR3 region represented by SEQ ID NO: 27, and a light chain variable region including a CDR1 region represented by SEQ ID NO: 28, a CDR2 region represented by SEQ ID NO: 29 and a CDR3 region represented by SEQ ID NO: 30;
6) a heavy chain variable region including a CDR1 region represented by SEQ ID NO: 31, a CDR2 region represented by SEQ ID NO: 32 and a CDR3 region represented by SEQ ID NO: 33, and a light chain variable region including a CDR1 region represented by SEQ ID NO: 34, a CDR2 region represented by SEQ ID NO: 35 and a CDR3 region represented by SEQ ID NO: 36;
7) a heavy chain variable region including a CDR1 region represented by SEQ ID NO: 37, a CDR2 region represented by SEQ ID NO: 38 and a CDR3 region represented by SEQ ID NO: 39, and a light chain variable region including a CDR1 region represented by SEQ ID NO: 40, a CDR2 region represented by SEQ ID NO: 41 and a CDR3 region represented by SEQ ID NO: 42;
8) a heavy chain variable region including a CDR1 region represented by SEQ ID NO: 43, a CDR2 region represented by SEQ ID NO: 44 and a CDR3 region represented by SEQ ID NO: 45, and a light chain variable region including a CDR1 region represented by SEQ ID NO: 46, a CDR2 region represented by SEQ ID NO: 47 and a CDR3 region represented by SEQ ID NO: 48; and
9) a heavy chain variable region including a CDR1 region represented by SEQ ID NO: 49, a CDR2 region represented by SEQ ID NO: 50 and a CDR3 region represented by SEQ ID NO: 51, and a light chain variable region including a CDR1 region represented by SEQ ID NO: 52, a CDR2 region represented by SEQ ID NO: 53 and a CDR3 region represented by SEQ ID NO: 54.

In an exemplary embodiment of the present disclosure, the antigen-binding fragment may be selected from a group consisting of Fab, Fab′, F(ab′)₂, scFv, Fv, dsFv, a diabody, Fd and Fď′.

In addition, the present disclosure relates to a polynucleotide encoding the monoclonal antibody or an antigen-binding fragment thereof.

In an exemplary embodiment of the present disclosure, the polynucleotide may be represented by any sequence selected from SEQ ID NO: 73 to SEQ ID NO: 81.

In addition, the present disclosure relates to an expression vector including the polynucleotide.

In addition, the present disclosure relates to a transgenic organism transformed with the expression vector.

In addition, the present disclosure relates to a pharmaceutical composition for preventing or treating cancer in which cathepsin Z is overexpressed, which contains the monoclonal antibody or an antigen-binding fragment thereof, a polynucleotide encoding the monoclonal antibody or an antigen-binding fragment thereof, or an expression vector including the polynucleotide as an active ingredient.

In addition, the present disclosure relates to a method for preventing or treating cancer, which includes a step of administering the monoclonal antibody or an antigen-binding fragment thereof, a polynucleotide encoding the monoclonal antibody or an antigen-binding fragment thereof, or an expression vector including the polynucleotide to a subject in need thereof.

In an exemplary embodiment of the present disclosure, the cancer may be brain cancer.

In an exemplary embodiment of the present disclosure, the brain cancer may malignant brain cancer or brain tumor.

In an exemplary embodiment of the present disclosure, the malignant brain cancer may be glioblastoma or glioblastoma multiforme.

In an exemplary embodiment of the present disclosure, the brain tumor may be anaplastic astrocytoma.

In addition, the present disclosure relates to a composition for diagnosing cancer in which cathepsin Z is overexpressed, which contains the monoclonal antibody or an antigen-binding fragment thereof, a polynucleotide encoding the monoclonal antibody or an antigen-binding fragment thereof, or an expression vector including the polynucleotide.

In addition, the present disclosure relates to a kit for diagnosing cancer in which cathepsin Z is overexpressed, which includes the composition and an instruction.

In addition, the present disclosure relates to a method for providing information necessary for diagnosis of cancer in which cathepsin Z is overexpressed, which includes: (a) a step of contacting the monoclonal antibody or an antigen-binding fragment thereof with a biological sample isolated from a subject suspected of glioblastoma; (b) a step of measuring the expression level of the cathepsin Z protein bound to the monoclonal antibody or the antigen-binding fragment thereof in the biological sample through formation of an antigen-antibody complex; and (c) a step of diagnosing as cancer in which cathepsin Z is overexpressed if the expression level of the cathepsin Z protein measured in the step (b) is higher as compared to a control group.

### Advantageous Effects

A monoclonal antibody specifically binding to cathepsin Z of the present disclosure, or an antigen-binding fragment thereof specifically binds to CtsZ, and thus can inhibit in vivo tumorigenesis of cancer in which CtsZ is overexpressed, particularly, brain cancer, more particularly, mesenchymal brain cancer stem cells. Therefore, the present disclosure can be usefully used for treatment of patients with cancer in which CtsZ is overexpressed, specifically patients with mesenchymal brain cancer, more specifically patients with glioblastoma. In addition, the monoclonal antibody of the present disclosure or an antigen-binding fragment thereof, which specifically binds to cathepsin Z, allows diagnosis of cancer in which CtsZ is overexpressed, specifically brain cancer, more specifically glioblastoma.

### BRIEF DESCRIPTION OF DRAWINGS

FIG. 1 schematically shows the structure of CtsZ according to an exemplary embodiment of the present disclosure.

FIG. 2 shows the RT-PCR result of cDNA of human CtsZ according to an exemplary embodiment of the present disclosure.

FIG. 3 shows a result of purifying a medium to which CtsZ is released with a Ni-NTA affinity column according to an exemplary embodiment of the present disclosure.

FIG. 4 shows a result of investigating the expression of CtsZ by western blot using an anti-6X his tag antibody according to an exemplary embodiment of the present disclosure.

FIG. 5 shows the binding affinity of integrin αVβ3 which binds to purified CtsZ according to an exemplary embodiment of the present disclosure.

FIG. 6 schematically shows the binding of a CNBr-activated resin and CtsZ and the result of the binding according to an exemplary embodiment of the present disclosure.

FIG. 7 shows the amino acid sequence of an OPAL phage display library according to an exemplary embodiment of the present disclosure.

FIG. 8 schematically shows a biopanning procedure according to an exemplary embodiment of the present disclosure.

FIG. 9 and FIG. 10 show input, output and output/input phage titers for each round during first biopanning according to an exemplary embodiment of the present disclosure.

FIG. 11a shows the dot blot analysis result for the 3 round output of first biopanning according to an exemplary embodiment of the present disclosure, FIG. 11b shows a result of performing plate binding test to investigate whether a purified CtsZ antigen binds to a plate according to an exemplary embodiment of the present disclosure, and FIG. 11c shows the binding strength of CtsZ for the 3 round output of first biopanning according to an exemplary embodiment of the present disclosure.

FIG. 12a shows the dot blot analysis result for the 4 round output of first biopanning according to an exemplary embodiment of the present disclosure, and FIG. 12b shows the binding strength of CtsZ for the 4 round output of first biopanning according to an exemplary embodiment of the present disclosure.

FIG. 13 and FIG. 14 show input, output and output/input phage titers for each round during second biopanning according to an exemplary embodiment of the present disclosure.

FIG. 15a shows the dot blot analysis result for the 4 round output of second biopanning according to an exemplary embodiment of the present disclosure, and FIG. 15b shows the binding strength of CtsZ for the 4 round output of second biopanning according to an exemplary embodiment of the present disclosure.

FIG. 16a and FIG. 16b show the scFv ELISA result for each clone for the 3 round of first biopanning and each clone for the 4 round of second biopanning according to an exemplary embodiment of the present disclosure.

FIG. 17a shows the dot blot analysis result for the 5 round output of second biopanning according to an exemplary embodiment of the present disclosure, and FIG. 17b shows the binding strength of CtsZ for the 5 round output of second biopanning according to an exemplary embodiment of the present disclosure.

FIG. 18 shows the scFv ELISA result for each clone for the 5 round of second biopanning according to an exemplary embodiment of the present disclosure.

FIGS. 19a to 19h show a result of analyzing the sequences of scFvs specific for 39 types of CtsZ screened according to an exemplary embodiment of the present disclosure.

FIG. 20 shows the specific sequences of 12 AE subtypes out of 39 clones for scFvs specific for 96 types of CtsZ screened according to an exemplary embodiment of the present disclosure, excluding enriched sequences.

FIG. 21 shows the specific sequences of 10 BE subtypes out of 39 clones for scFvs specific for 96 types of CtsZ screened according to an exemplary embodiment of the present disclosure, excluding enriched sequences.

FIG. 22 shows the specific sequences of 10 CF subtypes out of 39 clones for scFvs specific for 96 types of CtsZ screened according to an exemplary embodiment of the present disclosure, excluding enriched sequences.

FIG. 23 shows the specific sequences of 7 DF subtypes out of 39 clones for scFvs specific for 96 types of CtsZ screened according to an exemplary embodiment of the present disclosure, excluding enriched sequences.

FIG. 24 shows the ELISA analysis result showing the binding strength of CtsZ-specific scFvs screened according to an exemplary embodiment of the present disclosure for CtsZ, integrin αVβ3 and fibronectin, respectively.

FIG. 25 shows the specific sequences of scFvs specific for 9 types of CtsZ finally screened according to an exemplary embodiment of the present disclosure.

FIG. 26 shows the purity and K_{D} values of scFvs specific for 9 types of CtsZ finally screened according to an exemplary embodiment of the present disclosure determined by SDS-PAGE analysis.

FIG. 27 shows a histogram of treating 83NS cells with an anti-CtsZ antibody of the present disclosure and investigating cell death by FACS (cell cycle) analysis according to an exemplary embodiment of the present disclosure.

FIG. 28 shows a quantitative graph of treating 83NS cells with an anti-CtsZ antibody of the present disclosure and investigating cell death by FACS (cell cycle) analysis according to an exemplary embodiment of the present disclosure.

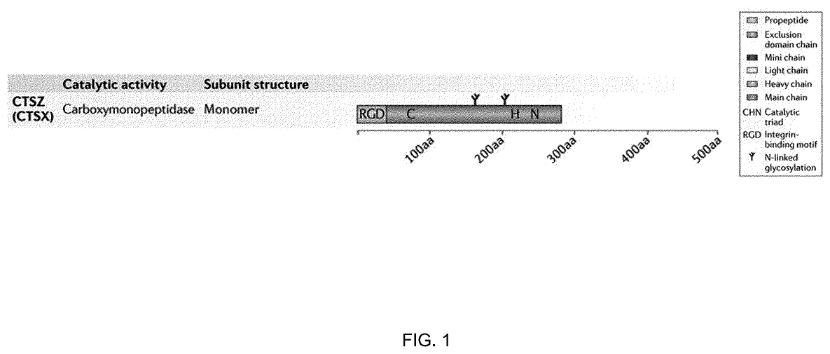

### BEST MODE

Hereinafter, the present disclosure is described in detail.

The present disclosure provides a monoclonal antibody specifically binding to cathepsin Z, or an antigen-binding fragment thereof. The CtsZ is specific for cancer in which cathepsin Z is overexpressed, specifically brain cancer, more specifically malignant brain cancer or brain tumor, more specifically glioblastoma (e.g., specifically expressed in glioblastoma).

In the present disclosure, the term “cathepsin” is an enzyme present in lysosomes, which induces the degradation of damaged proteins via the endosome-lysosome pathway. Cathepsin is classified into cysteine, aspartate and serine cathepsin depending on the activated catalytic residue site. 11 isozymes of cathepsin (B, C, F, H, K, L, O, S, V, Z and W) are known. It is known that cathepsin is expressed highly in various types of tumor, exhibits inversely proportional relationship with the survival rate of patients, and plays an important role in tumorigenesis and tumor development. Since cathepsin Z (hereinafter, referred to as ‘CtsZ’) has an RGD peptide sequence in a pro-domain unlike other cathepsin family members, it binds to the integrins of cancer cells and nearby cells through autocrine and paracrine signaling and delivers tumor-promoting signals to cancer cells, after being secreted out of cells.

In the present disclosure, “cathepsin Z” plays an important role in the microenvironment of diseases since it retains activity stably even under neutral pH environments, unlike other cysteine cathepsins, and its expression is limited in specific tissues. In immune cells, CtsZ regulates immune response through activation of MHC class II, which is important for antigen presentation, by degrading the invariant chain. And, in cancer cells, CtsZ increases the growth of cancer cells by controlling metastasis and angiogenesis. Particularly, in cancer cells, the inhibited expression and binding of CtsZ not only inhibits angiogenesis, but also reduces the growth and metastasis of cancer cells and induces cell death. Although it is reported that CtsZ shows increased expression in liver cancer, colorectal cancer, prostate cancer, etc. and in inflammations caused by Helicobacter infection in the stomach, and plays an important role in tumor malignancy in a pancreatic cancer model, its role in malignant brain cancer is not known well (Nagler D K et al., Prostate. 2004 Jul. 1; 60 (2): 109-19./Wang J et al., PLOS One. 2011; 6 (9): e24967./Vizin T et al., BMC Cancer. 2014 Apr. 13; 14:259./Krueger S et al., J Pathol. 2005 September; 207 (1): 32-42./Bernhardt A et al., J Biol Chem. 2010 Oct. 29; 285 (44): 33691-700./Akkri L et al. Genes Dev. 2014 Oct. 1; 28 (19): 2134-50). Considering that CtsZ plays a key role in the tumorigenesis and development of epithelial cancers, it is thought that CtsZ, the expression of which is remarkably increased in MES brain cancer, is highly valuable as an important therapeutic target for MES brain cancer which is highly resistant to treatment.

The inventors of the present disclosure have identified an antibody that specifically recognizes CtsZ, and have elucidated its heavy chain variable region amino acid sequence, light chain variable region amino acid sequence, and coding nucleotide sequence.

In addition, in a specific exemplary embodiment, the present disclosure provides a monoclonal antibody specifically binding to CtsZ, or an antigen-binding fragment thereof, so that cancer in which cathepsin Z is overexpressed, specifically one or more cancer selected from brain cancer, liver cancer, colorectal cancer and prostate cancer, more specifically malignant brain cancer or brain tumor, more specifically glioblastoma, glioblastoma multiforme or anaplastic astrocytoma, more specifically glioblastoma cells and/or tissues, can be diagnosed accurately by specifically detecting CtsZ.

In the present disclosure, the term “antibody” refers to a protein molecule acting as a receptor that specifically recognizes an antigen, including an immunoglobulin molecule which is immunologically reactive for a specific antigen, and includes not only a polyclonal antibody, a monoclonal antibody and a whole antibody, but also an antigen-binding fragment (antibody fragment) of the antibody molecule. In addition, the term also includes a chimeric antibody, a humanized antibody, a bivalent or bispecific molecule (e.g., a bispecific antibody), a diabody, a triabody and a tetrabody.

The “whole antibody” has two full-length light chains and two full-length heavy chains, and each of the light chains is linked to a heavy chain by a disulfide bond. The whole antibody includes IgA, IgD, IgE, IgM and IgG, and IgG includes IgG1, IgG2, IgG3 and IgG4 as subtypes.

In the present disclosure, the term “antigen-binding fragment of an antibody” refers to a fragment in the whole antibody molecule retaining the ability of antigen-antibody binding, and includes Fab, Fab′, F(ab′)₂, scFv, Fv, dsFv, a diabody, Fd, Fď′, etc. Fab has a structure consisting of a light chain, a heavy chain variable region, a light chain constant region, and a first heavy chain constant region (C_{H}1 domain) and it includes one antigen-binding site. Fab′ differs from Fab in that it has a hinge region including at least one cysteine residue in the C-terminus of the heavy chain C_{H}1 domain. An F(ab′)₂ antibody is formed by a disulfide bond between cysteine residues in the hinge region of Fab′. Fv (variable fragment) refers to a minimum antibody fragment having only a heavy chain variable region and a light chain variable region. A double-chain Fv (dsFv) has a structure wherein a heavy chain variable region is linked to a light chain variable region by a disulfide bond, and a single-chain Fv (scFv) has a structure where a heavy chain variable region is covalently linked to a light chain variable region by a peptide linker. A diabody refers to a complex of two or more polypeptide chains or proteins, each including at least one V_{L} and V_{H} domains or fragments thereof, wherein both domains are included within a single polypeptide chain. In some exemplary embodiments, the diabody includes a molecule including an Fc or hinge-Fc domain. In the complex, the polypeptide chains may be the same or different. That is, the diabody may be a monopolymer or a heteropolymer.

These antibody fragments can be obtained using a protease (e.g., Fab fragments can be obtained by cleaving a whole antibody with papain, and F(ab′)₂ fragments can be obtained by cleaving a whole antibody with pepsin), specifically by genetic recombination technology, although not being limited thereto.

In the present disclosure, the term “heavy chain” includes a full-length heavy chain including a variable region domain V_{H} including an amino acid sequence having a variable region sequence sufficient to confer specificity to an antigen, and three constant region domains, C_{H}1, C_{H}2 and C_{H}3, and a fragment thereof. In addition, the term “light chain” includes a full-length light chain including a variable region domain V_{L} including an amino acid sequence having a variable region sequence sufficient to confer specificity to an antigen, and a constant region domain C_{L}, and a fragment thereof.

In the present disclosure, the term “monoclonal antibody” refers to an antibody molecule having a single molecular composition, obtained from a population of substantially identical antibodies. The monoclonal antibody exhibits a single binding specificity and affinity for a particular epitope. Typically, an immunoglobulin has a heavy chain and a light chain, and each heavy and light chain includes a constant region and a variable region (regions are also referred to as “domains”). The light and heavy chain variable regions include three variable regions called complementarity-determining regions (“CDRs”) and four framework regions (FRs). The CDRs are primarily responsible for binding to the epitope of an antigen. The CDRs for each chain are typically CDR1, CDR2 and CDR3, numbered consecutively from the N-terminus, and are also typically identified by the chain in which the particular CDR is located.

In the present specification, the term “CDR (complementarity-determining region)” refers to the amino acid sequence of a hypervariable region of a heavy chain and a light chain of an immunoglobulin (Kabat et al. Sequences of Proteins of Immunological Interest, 4th Ed., U.S. Department of Health and Human Services, National Institutes of Health (1987)). Each of the heavy chain (CDRH1, CDRH2 and CDRH3) and the light chain (CDRL1, CDRL2 and CDRL3) includes three CDRs, and the CDRs provide major contact residues for binding of an antibody to an antigen or an epitope.

Meanwhile, the monoclonal antibody may be a chimeric antibody or a humanized antibody with reduced immunogenicity for application to the human body, or a human antibody.

In the present disclosure, the term “chimeric antibody” refers to an antibody in which the variable region of an antibody from a non-human species such as mouse, chicken, etc. is combined with the constant region of a human antibody by recombinant DNA technology. The chimeric antibody may be used clinically since immune response is greatly improved for human as compared to the antibodies from non-human species such as mouse, chicken, etc.

In the present disclosure, the term “humanized antibody” refers to an antibody in which all or part of the CDR sequence of a monoclonal antibody from a non-human species such as mouse, chicken, etc. is inserted into a human antibody. For example, the CDRs of a chicken or mouse monoclonal antibody may be combined with an FR derived from a human antibody to prepare a humanized variable region, and it may be combined with the constant regions of a desired human antibody, although not being limited thereto. In addition, since affinity for a humanized antibody is reduced when only the CDRs derived from mouse or chicken are introduced, the FR amino acid residue that may affect the 3D structure of the CDRs may be substituted with the amino acid of the mouse or chicken antibody to enhance affinity, although not being limited thereto.

In the present disclosure, the term “monoclonal antibody specifically binding to cathepsin Z” refers to an antibody that can specifically bind to CtsZ, and may be used interchangeably with an anti-CtsZ antibody in the present disclosure. The monoclonal antibody specifically binding to the CtsZ protein includes any monoclonal antibody which binds to CtsZ and inhibits the biological activity of CtsZ, without limitation. In addition, as described above, the monoclonal antibody may be a whole antibody or an antigen-binding fragment, and may be a chimeric antibody or a humanized antibody, although not being limited thereto. In addition, since the monoclonal antibody of the present disclosure specifically binds to CtsZ and inhibits its biological activity by inhibiting signaling by CtsZ, it may be usefully used for preventing or treating a disease, such as cancer in which CtsZ is involved, specifically cancer in which cathepsin Z is overexpressed, specifically one or more cancer selected from brain cancer, liver cancer, colorectal cancer and prostate cancer, more specifically malignant brain cancer or brain tumor, more specifically glioblastoma, glioblastoma multiforme or anaplastic astrocytoma, more specifically glioblastoma. In addition, since the overexpression of CtsZ is reported in one or more cancer selected from brain cancer, liver cancer, colorectal cancer and prostate cancer, specifically malignant brain cancer or brain tumor, more specifically glioblastoma, glioblastoma multiforme or anaplastic astrocytoma, more specifically glioblastoma, the antibody of the present disclosure that can specifically bind to CtsZ has the ability of diagnosing cancer in which cathepsin Z is overexpressed, specifically one or more cancer selected from brain cancer, liver cancer, colorectal cancer and prostate cancer, more specifically malignant brain cancer or brain tumor, more specifically glioblastoma, glioblastoma multiforme or anaplastic astrocytoma, more specifically glioblastoma, with high sensitivity and specificity, and thus can be usefully used for diagnosis of the cancer. In an exemplary embodiment of the present disclosure, an antigen-binding fragment specifically binding to CtsZ may be prepared using CtsZ as an antigen protein.

The monoclonal antibody specifically binding to CtsZ, or an antigen-binding fragment thereof may include any heavy chain variable region and light chain variable region selected from a group consisting of 1) to 9), although not being limited thereto:
1) a heavy chain variable region including a CDR1 region represented by SEQ ID NO: 1, a CDR2 region represented by SEQ ID NO: 2 and a CDR3 region represented by SEQ ID NO: 3, and a light chain variable region including a CDR1 region represented by SEQ ID NO: 4, a CDR2 region represented by SEQ ID NO: 5 and a CDR3 region represented by SEQ ID NO: 6;
2) a heavy chain variable region including a CDR1 region represented by SEQ ID NO: 7, a CDR2 region represented by SEQ ID NO: 8 and a CDR3 region represented by SEQ ID NO: 9, and a light chain variable region including a CDR1 region represented by SEQ ID NO: 10, a CDR2 region represented by SEQ ID NO: 11 and a CDR3 region represented by SEQ ID NO: 12;
3) a heavy chain variable region including a CDR1 region represented by SEQ ID NO: 13, a CDR2 region represented by SEQ ID NO: 14 and a CDR3 region represented by SEQ ID NO: 15, and a light chain variable region including a CDR1 region represented by SEQ ID NO: 16, a CDR2 region represented by SEQ ID NO: 17 and a CDR3 region represented by SEQ ID NO: 18;
4) a heavy chain variable region including a CDR1 region represented by SEQ ID NO: 19, a CDR2 region represented by SEQ ID NO: 20 and a CDR3 region represented by SEQ ID NO: 21, and a light chain variable region including a CDR1 region represented by SEQ ID NO: 22, a CDR2 region represented by SEQ ID NO: 23 and a CDR3 region represented by SEQ ID NO: 24;
5) a heavy chain variable region including a CDR1 region represented by SEQ ID NO: 25, a CDR2 region represented by SEQ ID NO: 26 and a CDR3 region represented by SEQ ID NO: 27, and a light chain variable region including a CDR1 region represented by SEQ ID NO: 28, a CDR2 region represented by SEQ ID NO: 29 and a CDR3 region represented by SEQ ID NO: 30;
6) a heavy chain variable region including a CDR1 region represented by SEQ ID NO: 31, a CDR2 region represented by SEQ ID NO: 32 and a CDR3 region represented by SEQ ID NO: 33, and a light chain variable region including a CDR1 region represented by SEQ ID NO: 34, a CDR2 region represented by SEQ ID NO: 35 and a CDR3 region represented by SEQ ID NO: 36;
7) a heavy chain variable region including a CDR1 region represented by SEQ ID NO: 37, a CDR2 region represented by SEQ ID NO: 38 and a CDR3 region represented by SEQ ID NO: 39, and a light chain variable region including a CDR1 region represented by SEQ ID NO: 40, a CDR2 region represented by SEQ ID NO: 41 and a CDR3 region represented by SEQ ID NO: 42;
8) a heavy chain variable region including a CDR1 region represented by SEQ ID NO: 43, a CDR2 region represented by SEQ ID NO: 44 and a CDR3 region represented by SEQ ID NO: 45, and a light chain variable region including a CDR1 region represented by SEQ ID NO: 46, a CDR2 region represented by SEQ ID NO: 47 and a CDR3 region represented by SEQ ID NO: 48; and
9) a heavy chain variable region including a CDR1 region represented by SEQ ID NO: 49, a CDR2 region represented by SEQ ID NO: 50 and a CDR3 region represented by SEQ ID NO: 51, and a light chain variable region including a CDR1 region represented by SEQ ID NO: 52, a CDR2 region represented by SEQ ID NO: 53 and a CDR3 region represented by SEQ ID NO: 54.

In an exemplary embodiment of the present disclosure, the antigen-binding fragment may specifically be Fab, Fab′, F(ab′)₂, scFv, Fv, dsFv, diabody, Fd, Fd′, a light chain or a heavy chain including the CDR region of the present disclosure, or be a variable domain including the CDR region of the present disclosure, although not being limited thereto.

In another exemplary embodiment of the present disclosure, the monoclonal antibody or an antigen-binding fragment thereof may specifically include a heavy chain variable region and a light chain variable region selected from a group consisting of a heavy chain variable region including a polypeptide sequence represented by SEQ ID NO: 55 and a light chain variable region polypeptide sequence represented by SEQ ID NO: 56; a heavy chain variable region including a polypeptide sequence represented by SEQ ID NO: 57 and a light chain variable region including a polypeptide sequence represented by SEQ ID NO: 58; a heavy chain variable region including a polypeptide sequence represented by SEQ ID NO: 59 and a light chain variable region including a polypeptide sequence represented by SEQ ID NO: 60; a heavy chain variable region including a polypeptide sequence represented by SEQ ID NO: 61 and a light chain variable region including a polypeptide sequence represented by SEQ ID NO: 62; a heavy chain variable region including a polypeptide sequence represented by SEQ ID NO: 63 and a light chain variable region including a polypeptide sequence represented by SEQ ID NO: 64; a heavy chain variable region including a polypeptide sequence represented by SEQ ID NO: 65 and a light chain variable region including a polypeptide sequence represented by SEQ ID NO: 66; a heavy chain variable region including a polypeptide sequence represented by SEQ ID NO: 67 and a light chain variable region including a polypeptide sequence represented by SEQ ID NO: 68; a heavy chain variable region including a polypeptide sequence represented by SEQ ID NO: 69 and a light chain variable region including a polypeptide sequence represented by SEQ ID NO: 70; and a heavy chain variable region including a polypeptide sequence represented by SEQ ID NO: 71 and a light chain variable region including a polypeptide sequence represented by SEQ ID NO: 72.

All variants of the antibody of the present disclosure or an antigen-binding fragment thereof obtained through one or more of substitution, deletion, inversion, translocation, etc. of the antibodies described above having the effect to be achieved by the present disclosure are also included in the scope of the present disclosure.

The antibody of the present disclosure or an antigen-binding fragment thereof may exhibit high apoptotic activity and inhibit tumorigenesis for cancer in which cathepsin Z is overexpressed, specifically one or more cancer selected from brain cancer, liver cancer, colorectal cancer and prostate cancer, more specifically malignant brain cancer or brain tumor, more specifically glioblastoma, glioblastoma multiforme or anaplastic astrocytoma, more specifically glioblastoma.

According to an exemplary embodiment of the present disclosure, the monoclonal antibody specifically binding to CtsZ of the present disclosure exhibits high apoptotic activity for mesenchymal glioblastoma stem cells.

In addition, the present disclosure provides a polynucleotide encoding the monoclonal antibody or an antigen-binding fragment.

The polynucleotide may be a sequence selected from a group consisting of SEQ ID NO: 73 to SEQ ID NO: 81, although not being limited thereto.

In another aspect, the present disclosure provides an expression vector including the polynucleotide, and a transgenic organism to which the vector is introduced.

The expression vector including the polynucleotide encoding the monoclonal antibody provided in the present disclosure may be a vector that can replicate and/or express the polynucleotide in a eukaryotic or prokaryotic cell including a mammalian cell (e.g., human, monkey, rabbit, rat, hamster or mouse cell), a plant cell, a yeast cell, an insect cell or a bacterial cell (e.g., E. coli, etc.), although not being limited thereto. Specifically, it may be a vector which is operably linked to a suitable promoter, so that the polynucleotide can be expressed in a host cell, and includes at least one selection marker. For example, the polynucleotide may be introduced in a phage, a plasmid, a cosmid, a minichromosome, a viral vector, a retroviral vector, etc.

The expression vector including the polynucleotide encoding the monoclonal antibody may be an expression vector including a polynucleotide encoding the heavy chain or light chain of the monoclonal antibody, or an expression vector including a polynucleotide encoding the heavy chain and the light chain.

The transgenic organism to which the expression vector is introduced provided in the present disclosure may be a bacterial cell such as E. coli, Streptomyces, Salmonella typhimurium, etc.; a yeast cell; a fungal cell such as Pichia pastoris, etc.; an insect cell such as Drosophila, Spodoptera Sf9 cell, etc.; an animal cell such as CHO (Chinese hamster ovary) cell, SP2/0 (mouse myeloma) cell, human lymphoblastoid cell, COS cell, NSO (mouse myeloma) cell, 293T cell, bow melanoma cell, HT-1080, BHK (baby hamster kidney) cell, HEK (human embryonic kidney) cell, PERC.6 (human retinal) cell, etc.; or a plant cell, which has been transformed as the expression vector is introduced, although not being limited thereto.

In the present disclosure, the term “introduction” refers to a method of delivering a vector including the polynucleotide encoding the monoclonal antibody into a host cell. The introduction may be performed by various methods known in the art, such as calcium phosphate-DNA coprecipitation, DEAE-dextran-mediated transfection, Polybrene-mediated transfection, electroporation, microinjection, liposome fusion, Lipofectamine transfection, protoplast fusion, etc. In addition, transduction refers to delivery of a target material into a cell using a virus particle via infection. Additionally, the vector may be introduced into a host cell via gene bombardment, etc. In the present disclosure, introduction may be used interchangeably with transfection.

Specifically, the host cell of the present disclosure may be an ‘isolated’ host cell.

In another aspect, the present disclosure provides a pharmaceutical composition for preventing or treating cancer in which cathepsin Z is overexpressed, which contains the monoclonal antibody or an antigen-binding fragment thereof, a polynucleotide encoding the monoclonal antibody or an antigen-binding fragment thereof, or an expression vector including the polynucleotide as an active ingredient.

The description of “monoclonal antibody or an antigen-binding fragment thereof” and “cancer in which cathepsin Z is overexpressed” will be omitted to avoid unnecessary redundancy since they were already described in detail above.

In the present disclosure, the cancer may be one or more selected from brain cancer, liver cancer, colorectal cancer and prostate cancer.

In the present disclosure, the brain cancer may be malignant brain cancer or brain tumor.

In the present disclosure, the malignant brain cancer may be glioblastoma or glioblastoma multiforme.

In the present disclosure, the brain tumor may be anaplastic astrocytoma.

In the present disclosure, the term “prevention” may mean any action of inhibiting or delaying the onset of cancer in which cathepsin Z is overexpressed by administering the composition. In addition, in the present disclosure, the term “treatment” may mean any action of ameliorating or improving the symptoms of cancer in which cathepsin Z is overexpressed by administering the composition.

The pharmaceutical composition may further contain a pharmaceutically acceptable carrier.

In the present disclosure, the term “pharmaceutically acceptable carrier” refers to a carrier or a diluent which does not inhibit the biological activity and characteristics of an administered compound without irritating an organism. For a composition formulated as a liquid solution, one or more of saline, sterile water, Ringer's solution, buffered saline, albumin injection solution, dextrose solution, maltodextrin solution, glycerol and ethanol, which are sterile and biocompatible, may be used as the pharmaceutically acceptable carrier. If necessary, other common additives such as an antioxidant, a buffer, a bacteriostatic agent, etc. may be added. In addition, a diluent, a dispersant, a surfactant, a binder and a lubricant may be additionally added to prepare an injectable formulation such as an aqueous solution, a suspension, an emulsion, etc., a pill, a capsule, a granule or a tablet.

The pharmaceutical composition may be in the form of various oral or parenteral formulations. For formulation, a commonly used diluent or excipient such as a filler, an extender, a binder, a wetting agent, a disintegrant, a surfactant, etc. is used. Solid formulations for oral administration include a tablet, a pill, a powder, a granule, a capsule, etc., and the solid formulation is prepared by mixing one or more compound with at least one excipient, e.g., starch, calcium carbonate, sucrose, lactose, gelatin, etc. In addition to a simple excipient, a lubricant such as magnesium stearate, talc, etc. is also used. Liquid formulations for oral administration include a suspension, an internal solution, an emulsion, a syrup, etc. In addition to a commonly used simple diluent such as water and liquid paraffin, various excipients, e.g., a wetting agent, a sweetener, an aromatic, preservative, etc. may be included. Formulations for parenteral administration include a sterilized aqueous solution, a nonaqueous solution, suspension, emulsion, a freeze-dried formulation and a suppository. For the nonaqueous solution or suspension, propylene glycol, polyethylene glycol, vegetable oil such as olive oil, injectable ester such as ethyl oleate, etc. may be used. As a base of the suppository, Witepsol, macrogol, Tween 61, cocoa butter, laurin butter, glycerogelatin, etc. may be used.

The pharmaceutical composition may be in the form of any one selected from a group consisting of a tablet, a pill, a powder, a granule, a capsule, a suspension, an internal solution, an emulsion, a syrup, a sterilized aqueous solution, a nonaqueous solution, a freeze-dried formulation and a suppository.

The composition of the present disclosure may be administered in a pharmaceutically effective amount.

In the present disclosure, the term “pharmaceutically effective amount” refers to an amount sufficient to treat a disease at a reasonable benefit/risk ratio applicable to medical treatment, and an effective dosage level can be determined depending on factors including the subject type, severity, sex, age, cancer type, the activity of a drug, the sensitivity to the drug, administration time, administration route, excretion rate, duration of treatment, simultaneously used drugs, and other factors well known in the medical field. The composition of the present disclosure may be administered as an individual therapeutic agent or in combination with another therapeutic agent either sequentially or simultaneously, and may be administered as a single dose or as multiple dosages. It is important to administer an amount such that a maximum effect can be achieved with a minimum amount without side effects by taking all of the above-described factors into consideration, and such an amount can be readily determined by those skilled in the art.

In an example of the present disclosure, it was confirmed that the anti-CtsZ antibody of the present disclosure not only specifically binds to CtsZ but also induces cell death and inhibits in vivo tumorigenesis in cancer in which cathepsin Z is overexpressed, specifically one or more cancer selected from brain cancer, liver cancer, colorectal cancer and prostate cancer, more specifically malignant brain cancer or brain tumor, more specifically glioblastoma, glioblastoma multiforme or anaplastic astrocytoma, more specifically glioblastoma. Therefore, the pharmaceutical composition containing the antibody of the present disclosure can be usefully used for preventing or treating cancer in which cathepsin Z is overexpressed.

In another aspect, the present disclosure the present disclosure provides a method for treating cancer in which cathepsin Z is overexpressed, using the monoclonal antibody or an antigen-binding fragment thereof, a polynucleotide encoding the monoclonal antibody or an antigen-binding fragment thereof, or an expression vector including the polynucleotide.

The description of “monoclonal antibody or an antigen-binding fragment thereof” and “cancer in which cathepsin Z is overexpressed” will be omitted to avoid unnecessary redundancy since they were already described in detail above.

The method for treating cancer in which cathepsin Z is overexpressed may include a step of administering a pharmaceutical composition containing the monoclonal antibody of the present disclosure or an antigen-binding fragment thereof, a polynucleotide encoding the monoclonal antibody or an antigen-binding fragment thereof, or an expression vector including the polynucleotide, and a pharmaceutically acceptable carrier to a subject in need thereof, and the pharmaceutically acceptable carrier is the same ad described above. Specifically, the method for treating cancer in which cathepsin Z is overexpressed may include a step of administering a composition containing the monoclonal antibody, polynucleotide or expression vector of the present disclosure to a subject having cancer in which cathepsin Z is overexpressed.

The subject includes mammals including cow, pig, sheep, chicken, dog, human, etc., birds, etc. without limitation, as long as cancer in which cathepsin Z is overexpressed can be treated by administering the composition of the present disclosure.

The composition may be administered in a pharmaceutically effective amount as a single dose or as multiple dosages. The composition may be administered in the form of a liquid, a powder, an aerosol, a capsule, an enteric-coated tablet or capsule, or a suppository. The administration route includes intraperitoneal administration, intravenous administration, intramuscular administration, subcutaneous administration, endothelial administration, oral administration, topical administration, intranasal administration, intrapulmonary administration, rectal administration, etc., although not being limited thereto. For oral administration, an oral composition should be formulated such that the active drug is coated or protected from degradation in the stomach because the peptide is digested. In addition, the pharmaceutical composition may be administered by any device which can deliver an active substance to a target cell.

Since the pharmaceutical composition of the present disclosure contains the monoclonal antibody specifically binding to CtsZ of the present disclosure, the pharmaceutical composition containing the monoclonal antibody may be administered into the human body to treat cancer in which cathepsin Z is overexpressed by inhibiting or preventing the onset, proliferation or metastasis of cancer in which cathepsin Z is overexpressed.

In another aspect, the present disclosure provides a composition for diagnosing cancer in which cathepsin Z is overexpressed, which contains the monoclonal antibody or an antigen-binding fragment thereof, a polynucleotide encoding the monoclonal antibody or an antigen-binding fragment thereof, or an expression vector including the polynucleotide as an active ingredient.

The description of “monoclonal antibody or an antigen-binding fragment thereof” and “cancer in which cathepsin Z is overexpressed” will be omitted to avoid unnecessary redundancy since they were already described in detail above. In the present disclosure, the term “diagnosis” means the action of identifying the presence, degree (symptoms) and/or characteristics of a pathological condition. As used in the present specification, the “diagnosis of cancer in which cathepsin Z is overexpressed” means the action of identifying the pathological condition of cancer in which cathepsin Z is overexpressed, e.g., the onset of cancer in which cathepsin Z is overexpressed, the degree of cancer in which cathepsin Z is overexpressed, the location of cancer in which cathepsin Z is overexpressed, etc. For more accurate diagnosis, it is important to distinguish accurately and quickly the cells and/or tissues of cancer in which cathepsin Z is overexpressed from normal cells or normal tissues.

In a specific exemplary embodiment of the present disclosure, by providing the monoclonal antibody specifically binding to CtsZ, the present disclosure may allow accurate diagnosis of the cells and/or tissues of cancer in which cathepsin Z is overexpressed by specifically detecting CtsZ at the protein level. In addition, a diagnostic composition containing the monoclonal antibody specific for CtsZ of the present disclosure may be used to diagnose a disease associated with the expression or expression level of CtsZ or a disease mediated by CtsZ, e.g., cancer in which cathepsin Z is overexpressed.

In another aspect, the present disclosure the present disclosure provides a kit for diagnosing cancer in which cathepsin Z is overexpressed, which includes the composition for diagnosing cancer in which cathepsin Z is overexpressed.

The description of “monoclonal antibody or an antigen-binding fragment thereof” and “cancer in which cathepsin Z is overexpressed” will be omitted to avoid unnecessary redundancy since they were already described in detail above.

The kit for diagnosing cancer in which cathepsin Z is overexpressed may further include one or more composition, solution or device suitable for analysis. In addition, the kit for diagnosing cancer in which cathepsin Z is overexpressed may further include an instruction.

The present disclosure provides a means useful for measuring the expression and/or expression level of CtsZ by providing an antibody which specifically binds to CtsZ overexpressed in cancer in which cathepsin Z is overexpressed and, thus, can be usefully used to diagnose cancer in which cathepsin Z is overexpressed. In addition, the antibody may be used, when various labeling materials are bound, to visualize cancer in which cathepsin Z is overexpressed, particularly to provide more accurate information for identification of the presence or lesion site of cancer in which cathepsin Z is overexpressed, and morphological observation of cancer in which cathepsin Z is overexpressed, and may contribute to early diagnosis of cancer in which cathepsin Z is overexpressed with decreased rate of misdiagnosis. In addition, when a bioactive substance such as a drug, etc. is bound to the antibody, it may be used as a composition for delivering a bioactive substance for targeting cancer in which cathepsin Z is overexpressed since it can specifically deliver the bioactive substance to colorectal cancer.

In another aspect, the present disclosure provides a method for providing information necessary for diagnosis of cancer in which cathepsin Z is overexpressed, which includes: (a) a step of contacting the monoclonal antibody or an antigen-binding fragment thereof with a biological sample isolated from a subject suspected of cancer in which cathepsin Z is overexpressed; (b) a step of measuring the expression level of the cathepsin Z protein bound to the monoclonal antibody or the antigen-binding fragment thereof in the biological sample through formation of an antigen-antibody complex; and (c) a step of diagnosing as cancer in which cathepsin Z is overexpressed if the expression level of the cathepsin Z protein measured in the step (b) is higher as compared to a control group.

The monoclonal antibody, cancer in which cathepsin Z is overexpressed, subject and CtsZ are the same as described above. The method for providing information necessary for diagnosis of cancer in which cathepsin Z is overexpressed allows the measurement of the expression level of the CtsZ protein through reaction of the monoclonal antibody specific for CtsZ of the present disclosure with a biological sample isolated from a subject suspected of cancer in which cathepsin Z is overexpressed and formation of an antigen-antibody complex and, through this, may provide information for diagnosing cancer in which cathepsin Z is overexpressed. Since CtsZ is overexpressed in cancer cells in which cathepsin Z is overexpressed, the subject may be diagnosed as having cancer in which cathepsin Z is overexpressed when the expression level of CtsZ is higher as compared to a control group such as normal cells or tissues.

In the present disclosure, the term “biological sample” may encompass a tissue, a cell, whole blood, serum, plasma, a tissue autopsy sample (brain, skin, lymph node, spinal cord, etc.), saliva, urine, a cell culture supernatant, a disrupted eukaryotic cell, a bacterial expression system, etc., although not being limited thereto. The biological sample may be reacted with the antibody of the present disclosure with or without manipulation to identify the presence or expression level of the CtsZ protein, or to identify cancer in which cathepsin Z is overexpressed.

The expression level of the cathepsin Z protein may be measured by measuring the expression level of the cathepsin Z protein bound to the monoclonal antibody or antigen-binding fragment thereof through the formation of the antigen-antibody complex in the biological sample, although not being limited thereto.

In the present disclosure, the term “antigen-antibody complex” refers to a complex of the CtsZ protein antigen in a sample and the monoclonal antibody according to the present disclosure which recognizes the same. The formation of the antigen-antibody complex may be detected by any method selected from a group consisting of a colorimetric method, an electrochemical method, a fluorimetric method, luminometry, a particle counting method, visual assessment, and a scintillation counting method. However, various applications and adaptations are possible, without being necessarily limited thereto.

In the present disclosure, various labels may be used to detect the antigen-antibody complex. Specific examples may be selected from a group consisting of an enzyme, a fluorophore, a ligand, a luminophore, a microparticle and a radioisotope, although not being necessarily limited thereto.

As the detection label, the enzyme includes acetylcholinesterase, alkaline phosphatase, β-D-galactosidase, horseradish peroxidase, β-lactamase, etc., the fluorophore includes fluorescein, Eu³⁺, Eu³⁺ chelate or cryptate, etc., the ligand includes a biotin derivative, etc., the luminophore includes an acridinium ester, an isoluminol derivative, etc., the microparticle includes colloidal gold, colored latex, etc., and the radioisotope includes ⁵⁷Co, ³H, ¹²⁵I, ¹²⁵I-Bonton Hunter reagent, etc.

Specifically, the antigen-antibody complex may be detected by enzyme-linked immunosorbent assay (ELISA). The enzyme-linked immunosorbent assay (ELISA) includes various ELISA methods such as direct ELISA using a labeled antibody recognizing an antigen attached to a solid support, indirect ELISA using a labeled secondary antibody recognizing a capture antibody in a complex of an antibody and an antigen attached to a solid support, direct sandwich ELISA using another labeled antibody recognizing an antigen in a complex of an antibody and an antigen attached to a solid support, indirect sandwich ELISA using a labeled secondary antibody reacting with another antibody recognizing an antigen in a complex of an antibody and an antigen attached to a solid support and recognizing the antibody, etc.

The monoclonal antibody may have a detection label. When it has no detection label, it may be identified by treating with another antibody which can capture the monoclonal antibody and has a detection label.

In an example of the present disclosure, it was confirmed that the anti-CtsZ antibody of the present disclosure specifically recognizes CtsZ via an antigen-antibody reaction. Therefore, the antibody of the present disclosure can be usefully used to diagnose cancer in which cathepsin Z is overexpressed.

Hereinafter, the monoclonal antibody according to the present disclosure or an antigen-binding fragment thereof will be described in detail through examples.

### MODE FOR INVENTION

### Materials and Methods

### 1. Materials

DNA sequencing was performed by Bionics. Ni-NTA resin and HRP-conjugated streptavidin were purchased from Thermo Fisher Scientific (USA). A MiniTrap G-25 column, a PD-10 desalination column and CNBr-activated Sepharose 4B were purchased from Cytiva (USA). HRP-conjugated anti-6x his tag antibody and HRP-conjugated anti-human Fc antibody were purchased from Invitrogen (USA). Human Integrin αVβ3 was purchased from ACROBiosystems (USA). Human fibronectin and human integrin alpha V/CD51 biotinylated biotinylated antibody were purchased from R&D Systems (USA). HRP-conjugated anti-HA tag antibody was acquired from Bethyl (USA). Protein A resin was purchased from GenScript (USA).

### 2. Recombinant CtsZ Protein

The CtsZ gene was obtained by culturing human breast cancer cells (MDA-MB-231) in an FBS medium to 1.0×10⁶ cells/mL.

Specifically, pellets were obtained by centrifuging the cultured MDA-MB-231 cells and RNA was extracted using TRIzol. The cDNA of the MDA-MB-231 cells was synthesized by reverse transcription PCR using the extracted RNA as a template. Then, CtsZ gene PCR was conducted using the cDNA of the MDA-MB-231 cells as a template (Table 1 to Table 3). In addition, a Kozak sequence (GCCGCCACCCATGGG) was added to the N-terminus of CtsZ and a 6x-His tag was added to the C-terminus through CtsZ gene PCR. The amplified CtsZ gene was inserted into the Xbal and Xhol sites of pcDNA3.4. The pcDNA3.4-CtsZ plasmid was transfected into 30 mL of Expi-293FTM cells. After 20 hours of incubation, the cells were cultured for 6 days together with an induction agent. After the culturing was completed, the medium was centrifuged (3,500 g, 20 minutes, 4° C.) and filtered through a 0.45-μm syringe filter. The filtered medium was incubated at 4° C. for 2 hours with Ni-NTA resin equilibrated previously with 1×PBS (pH 7.4) and loaded into a disposable column. After washing with 10 RV (resin volume) of an equilibration buffer, the protein was eluted in PBS (pH 7.4) with 5 to 200 mM imidazole gradient. Finally, the eluate was buffer-exchanged with PBST (0.01% Tween-20) at pH 7.4 using a PD-10 desalination column. The purified CtsZ was stored at −80° C. until use. The primers used in the PCR are described in Table 1, and the PCR conditions are described in Table 2 and Table 3.

TABLE 1

| | Base sequence |
|---|---|
| Forward Primer | TATCAATCTAGAGCCGCCACCATGGCGAGGCGCGGGCCAG |
| Overhang | TATCAA |
| XbaI | TCTAGA |
| Kozak | GCCGCCACCATGG |
| Annealing site | CCATGGCGAGGCGCGGGCCAG |
| Reverse Primer | TATCAACTCGAGTCATTAGTGGTGATGGTGATGATGAACGAT |
| | GGGGTCCCCAAATGTACAGTGCTCCTC |
| Overhang | TATCAA |
| XhoI | CTCGAG |
| Stop codon | TCATTA |
| Annealing site | AACGATGGGGTCCCCAAATGTACAGTGCTCCTC |

TABLE 2

| PCR materials | Volume |
|---|---|
| Platinum ™ SuperFi II PCR master | 25 μL |
| mix (Invitrogen ™, 12368010) | |
| Primers (forward/reverse) | 2.5 μL/2.5 μL |
| cDNA template | 2 μL (2 μg) |
| Nuclease | 18 μL |
| Total | 50 μL |

TABLE 3

| PCR condition | Volume |
|---|---|
| Initial denaturation | 98° C., 30 s (1 cycle) |

| | | |
|---|---|---|
| Denaturation | 98° C., 10 s | 30 cycles |
| Annealing | 65° C., 10 s | 30 cycles |
| Extension | 72° C., 30 s | 30 cycles |

| | |
|---|---|
| Final extension | 72° C., 5 m (1 cycle) |

### 3. RNA Extraction Using TRIzol

The MDA-MB-231 cell pellets of 2 and 1 mL of TRIzol were mixed by vortexing in a 1.5 mL e-tube for 10 seconds. The mixture was incubated at room temperature for 5 minutes. After adding 200 μL of chloroform, the mixture was vortexed for 20 seconds. The mixture was incubated at room temperature for 3 minutes. After centrifugation for layer separation (13,000 g, 15 minutes, 4° C.), the transparent supernatant was transferred to a fresh e-tube. After adding 500 μL of isopropyl alcohol to the supernatant, followed by vortexing for 5 seconds, reaction was conducted at 25° C. for 10 minutes. After centrifuging at 12,000 rpm for 10 minutes to remove the supernatant, the pellets were resuspended in 1 mL of 70% ethanol. After the second centrifugation, the supernatant was removed and the pellets were dried for 10 minutes or longer. The completely dried pellets were dissolved in RNA-free water.

### 4. ELISA for Integrin αVβ3 Binding to Purified CtsZ

After coating 1×PBS (pH 7.4) containing 200 ng of CtsZ in each well of a 96-well high-binding plate at 4° C. for 16 hours, followed by washing twice with a washing buffer (20 mM Tris-HCl, 150 mM NaCl, 1 mM MnCl₂, 0.05% Tween-20), blocking was performed at room temperature for 2 hours using a blocking buffer (washing buffer containing 3% BSA). After washing twice with a washing buffer, binding was performed at room temperature for 2 hours using a dilution buffer (washing buffer containing 0.5% BSA) containing 200 nM, ⅓ eq. integrin αVβ3. After washing 4 times with a washing buffer, biotinylated anti-human integrin alpha V antibody (diluted to 1:100 with dilution buffer) was treated at room temperature for 1 hour. After washing 4 times with a washing buffer, HRP-conjugated streptavidin was treated at room temperature for 1 hour. After washing 4 times with a washing buffer, followed by addition of ultra TBM matrix, reaction was performed for 2 minutes and the reaction was stopped by adding 2 M H₂SO₄. K_{D} values were calculated from the absorbance at 450 nm using the Prism7 software.

### 5. Preparation of CtsZ-Conjugated Resin

The purified CtsZ was buffer-exchanged with 1×PBS (pH 7.4) using a previously equilibrated MiniTrap G-25 column. 0.3 mg of CtsZ and 0.1 mL of CNBr (cyanogen bromide)-activated resin washed previously were mixed at 4° C. for 16 hours in a spin column. After washing 3 times with 1×PBS, the remaining reactive groups were blocked with 0.1 M Tris-HCl (pH 8.0) for 2 hours. After washing 3 times with 1×PBS, a 10% (v/v) resin slurry was prepared by adding 0.9 mL of 1×PBS and stored at 4° C.

### 6. Biopanning for CNBr-Conjugated CtsZ

1 round biopanning was performed as follows. First, 100 UL of a resin solution (10 μL resin, 30 μg binding protein (CNBr-conjugated CtsZ)) was added to a centrifuge tube filter and centrifuged at 500 g for 30 seconds. Then, blocking was performed at room temperature for 2 hours with 700 μL of 3% BSA. After washing 3 times with 1x PBS (pH 7.4), incubation was performed at 4° C. for 16 hours with 200 μL of a phage library. An M13 phage library displaying scFv was provided by Professor Hyunbo Shim (Ewha Womans University, Korea) (Yang, H. Y., et al., Construction of a large synthetic human scFv library with six diversified CDRs and high functional diversity. Molecules and Cells, 2009. 27 (2): p. 225-235). For low-pH elution, 200 μL of an elution buffer (100 mM glycine pH 2.2, 1% BSA) was filled in a tube and incubated at room temperature for 10 minutes. Finally, a phage eluate was obtained through centrifugation (500 g, 30 seconds). For neutralization, 40 μL of 1 M Tris-HCl (pH 8.0) was added immediately.

For titration, 10 μL of the neutralized phage eluate was diluted sequentially using 1×PBS. E. coli ER2738 (OD₆₀₀=0.6-0.8) was infected with the diluted sample at room temperature for 30 minutes, and then sprayed onto an LB/agar plate containing carbenicillin.

For amplification of the phage, an SB medium containing 2 mL of E. coli ER2738 (OD₆₀₀=0.6-0.8, containing tetracycline) was infected with 200 μL of the neutralized phage eluate at 110 rpm and 37° C. for 1 hour. 2 mL of the infected cells were transferred to 10 mL of an SB medium and cultured at 37° C. and 180 rpm for 1 hour after adding 6 μL of carbenicillin (100 μg/mL). Then, 12 mL of the infected cells were transferred to 30 mL of an SB medium and cultured 30° C. and 180 rpm for 16 hours after adding 30 μL of carbenicillin and 42 μL of M13KO7 helper phage (2.4×10¹² PFU/mL) (NEB, UK). After centrifuging the cells at 3,500 g for 30 minutes, the supernatant was transferred to a fresh 50-mL conical tube. The supernatant was mixed with 1.6 g of PEG 8000 and 1.2 g of NaCl and dissolved completely by conducting reaction at 37° C. for 10 minutes. The mixture was incubated on ice for 4 hours. Then, the pellets obtained through centrifugation at 15,000 g for 1 hour were resuspended in 400 μL of a PBS buffer (1×PBS, 1% BSA). Finally, after centrifugation (15,000 g, 10 minutes), the recovered supernatant was filtered through a 0.22-μm PES syringe filter. The filtered supernatant was stored at 4° C. until use.

2 round and 3 round biopanning was performed under the same condition as the 1 round biopanning, but 4 round and 5 round biopanning was performed with not amplification process (From the 4 round biopanning, in-frame rate decreased rapidly below 5% when amplification the process was performed.).

7. Isolation of Periplasmic Fraction Containing scFv

The ER2738 infected with the phage was plated on an LB/agar plate containing carbenicillin. After filling 750 μL of an SB medium containing carbenicillin in a 96-deep well plate, each well was inoculated with a single colony. After incubation (4 hours, 37° C., 180 rpm), 75 L of 10 mM IPTG was added to each well. IPTG induction was conducted at 180 rpm and 30° C. for 16 hours. The induced cells were centrifuged (4,000 rpm, 20 minutes, 4° C.) and the deep well plate was immersed in ice after removing the supernatant.

The pellets were resuspended softly by adding to 200 μL of 1× TES buffer (20% sucrose, 50 mM Tris-HCl (pH 8.0), 1 mM EDTA) in ice. After inducing osmotic shock, 300 μL of 0.2x TES buffer was added to disrupt the outer membrane of the cells, and the pellets were incubated with ice for 30 minutes. The supernatant recovered after centrifugation (4000 rpm, 20 minutes, 4° C.) contained a periplasmic fraction containing scFv.

### 8. Dot Blot Assay

2 μL of the periplasmic fraction was transferred to an NC membrane using a 0.2-10 μL multipipette. In addition, 2 μL of PBS and 10-100 ng of scFv were transferred to a positive control group. The NC membrane was incubated with PBS containing 5% skim milk at room temperature for 1 hour. Then, it was washed 4 times with PBST (0.05% Tween-20), for 5 minutes each. For antibody reaction, HRP-conjugated anti-HA antibody was diluted in PBST (0.05%) to 1:3000 and incubated at room temperature for 1 hour. After washing 4 times with PBST (0.05% Tween-20), for 5 minutes each, followed by treatment with a chemiluminescent substrate, dots were detected using LAS4000.

### 9. ELISA of Periplasmic Fraction

100 μL of CtsZ (200 ng/well) was coated onto a 96-well high-binding plate at 4° C. for 16 hours. In the blocking step, 300 μL of 1×PBS (pH 7.4) containing 3% BSA was added to each well and incubation was performed at room temperature for 2 hours. After washing 4 times with 300 μL of 1×PBS and adding 100 μL of the periplasmic fraction (obtained in 7) to each well, incubation was performed at room temperature for 2 hours. After washing 4 times with 300 μL of PBST (0.05% Tween-20), 100 UL of HRP-conjugated anti-HA antibody diluted in PBST (0.05%) to 1:3000 was incubated at room temperature for 1 hour. After washing 4 times with 300 μL of PBST (0.05% Tween-20) and treating with 100 μL of an ultra TMB solution, reaction was conducted for 5 minutes and the reaction was terminated using 2 M H₂SO₄.

10. Screening of scFv Clones Specific for CtsZ

CtsZ-specific scFv clones were selected from 40 scFv clones by conducting negative selection ELISA. The nonspecific binding activity to integrin (ITG) and fibronectin (FN) was measured by ELISA. Specifically, in the antigen coating step of ELISA, 100 μL of Integrin aVß3 or fibronectin (200 ng/well) in 1×PBS (pH 7.4) was coated instead of CtsZ, and the following procedure was the same as that of the ELISA of periplasmic fraction (9).

11. Expression and Purification of Anti-CtsZ scFv

A pComb3X-scFv plasmid was transfected into TOP10F competent cells. The obtained colony was inoculated into 10 mL of an SB medium (containing carbenicillin and tetracycline) and precultured at 37° C. for 16 hours. The precultured cells were transferred to 500 mL of an SB medium (containing carbenicillin) and cultured at 37° C. to OD₆₀₀=0.6-0.8. After adding IPTG to a final concentration of 1 mM, IPTG induction was performed at 30° C. for 16 hours. Then, the cells were harvested through centrifugation (3,500 g, 20 minutes, 4° C.). After adding 35 mL of cold 1x TES buffer (20% sucrose, 50 mM Tris-HCl (pH 8.0), 1 mM EDTA) to the harvested cells, the mixture was resuspended. Then, after adding lysozymes to a final concentration of 1 mg/mL, the mixture was incubated on ice for 30 minutes. Subsequently, after inactivating remaining EDTA by adding 0.25 mL of 1 M MgCl₂, incubation was performed on ice for 10 minutes. Then, after performing centrifugation (10,000 g, 20 minutes, 4° C.) to recover a periplasmic fraction, the periplasmic fraction was mixed with 1 mL of a Ni-NTA resin previously equilibrated with 0.05% PBST for protein-resin binding (eq buffer:resin=1:1) and incubated at 4° C. for 1 hour. The protein-resin mixture was packed in a disposable column and then washed with 0.05% PBST to 20 RV (resin volume), and then with 0.05% PBST containing 5, 10 and 50 mM imidazole, respectively, to 10 RV. The protein eluate was eluted with 0.05% PBS containing 200 mM imidazole to 2.5 RV. Finally, the eluate was buffer-exchanged with 1×PBS (pH 7.4) using a PD-10 desalination column. The purified scFv clone was stored at −80° C. until use.

12. ELISA for Anti-CtsZ scFv Binding to CtsZ

100 μL of CtsZ (200 ng/well) dissolved in a coating buffer (1×PBS (pH 7.4)) was coated onto a 96-well high-binding plate at 4° C. for 16 hours. In the blocking step, incubation was performed at room temperature for 2 hours after adding 300 μL of a blocking buffer (washing buffer containing 3% BSA) to each well. After washing 4 times with 300 μL of a washing buffer (20 mM Tris, 150 mM NaCl, 1 mM MnCl₂, 0.05% Tween-20, pH 7.4), scFv clones purified to 700 nM, ¼ eq were bound at room temperature for 2 hours. After washing 4 times with 300 μL of a washing buffer, 100 μL of HRP-conjugated anti-HA antibody diluted to 1:3000 in a dilution buffer (washing buffer containing 0.5% BSA) was bound at room temperature for 1 hour. After washing 4 times with 300 μL of a washing buffer and treating with 100 μL of an ultra TMB solution, reaction was conducted for 2 minutes and the reaction was terminated using 2 M H₂SO₄. K_{D} values were calculated from the absorbance at 450 nm using the Prism7 software.

### 13. Expression and Purification of Anti-CtsZ IgG

In order to express anti-CtsZ IgG in mammalian cells, pcDNA3.4-IgH and pcDNA3.4-IgL plasmids were transfected into 30 mL of Expi-293FTM cells, and the cells were cultured at 37° C. for 20 hours under the condition of 120 rpm and 8% CO2. After treating with an inducer, the cells were cultured for 6 days. The culture medium was cultured through centrifugation (3,500 g, 20 minutes, 4° C.) and filtered using a 0.45-μm syringe filter. Then, for protein-resin binding, 0.5 mL of rProtein A resin (eq buffer:resin=1:1) equilibrated previously with 1×PBS (pH 7.4) was mixed with the medium and incubation was performed at 4° C. for 2 hours. The protein-resin mixture was packed in a disposable column and then washed with 1×PBS (pH 7.4) to 20 RV (resin volume), and then eluted with 2.33 mL of an IgG elution buffer. After the elution, the eluate was neutralized by immediately mixing with 233 μL of a neutralization buffer. The neutralized eluate was buffer-exchanged with 1×PBS (pH 7.4) using a PD-10 column. The purified anti-CtsZ IgG clones were stored at −80° C. until use.

### 14. ELISA for Anti-CtsZ IgG Binding to CtsZ

100 μL of CtsZ (200 ng/well) was coated onto a 96-well high-binding plate at 4° C. for 16 hours. In the blocking step, 300 μL of a blocking buffer (washing buffer containing 3% BSA) was added to each well and incubation was performed at room temperature for 2 hours. Purified IgG clones of 10 nM, ¼-fold (#1, 2, 3, 4, 6, 7), 600 nM, ¼-fold (#5, 8) or 2 μM, ¼-fold (#9) were bound using 300 μL of a washing buffer (20 mM Tris, 150 mM NaCl, 1 mM MnCl₂, 0.05% Tween-20 pH 7.4) at room temperature for 2 hours. After washing 4 times with 300 μL of a washing buffer, 100 μL of HPR-conjugated anti-human Fc antibody diluted to 1:2,500 with a dilution buffer (washing buffer containing 0.5% BSA) was bound at room temperature for 1 hour. After washing 4 times with 300 μL of a washing buffer and treating with 100 μL of an ultra TMB solution, reaction was conducted for 2 minutes and the reaction was terminated using 2 M H₂SO₄. K_{D} values were calculated from the absorbance at 450 nm using the Prism7 software.

### Test Example 1: Preparation and Screening of Anti-CtsZ Antibody

### 1-1. Expression and Purification of Human CtsZ

### 1-1-1. Expression and Purification of Human CtsZ

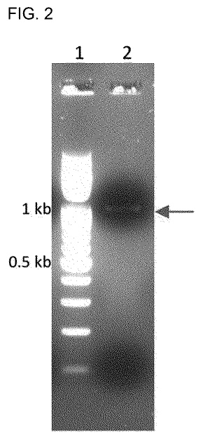

1) cDNA cloning and transfection: CtsZ is upregulated in MDA-MB-231 breast cancer cells. Therefore, RNA containing the CtsZ gene was obtained by extracting RNA from MDA-MB-231 cells. After synthesizing cDNA through RT-PCR, the Kozak sequence was added to the N-terminus to increase protein expression in mammalian cells, and a 6x-His tag was added to the C-terminus for purification during the amplification of the CtsZ gene (FIG. 2). Then, after cloning into a vector for expression in animal cells (pcDNA3.4), the vector was transfected into animal cells (Expi293F™ cells) (CtsZ is expressed in animal cells because it has two glycosylation sites.).

2) Isolation of medium: Because CtsZ has its own signal sequence, the CtsZ expressed in the animal cells is released to the medium. The medium was recovered by centrifugation at 3000 g for 30 minutes and then purified after filtering the cells through a 0.2-μm filter.

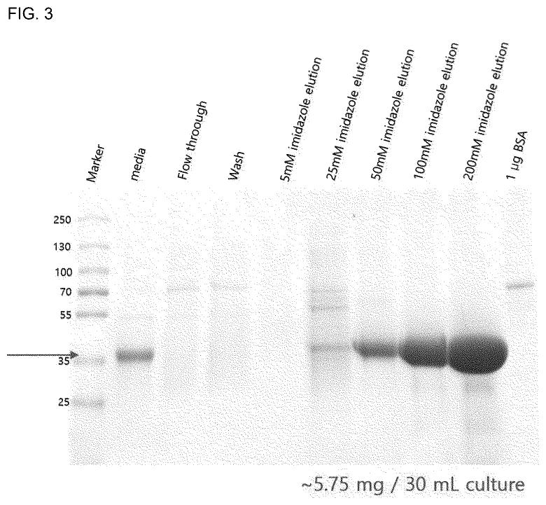

3) Purification: The medium to which the CtsZ was released was purified using a Ni-NTA affinity column (FIG. 3). Specifically, the medium to which the CtsZ was released was incubated with a Ni-NTA resin at 4° C. for 2 hours. After passing the flow-through through the column and washing with an imidazole-containing buffer, the CtsZ protein was obtained using an elution buffer (PBS (pH 7.4)+100 mM or 200 mM imidazole). Then, after buffer-exchanging with PBST (pH 7.4, 0.01% Tween-20) through a desalination column, the CtsZ protein was stored at −80° C.

### 1-1-2. SDS-PAGE and Western Blot Assays

The expression of CtsZ was identified by western blot using anti-6X his tag antibody.

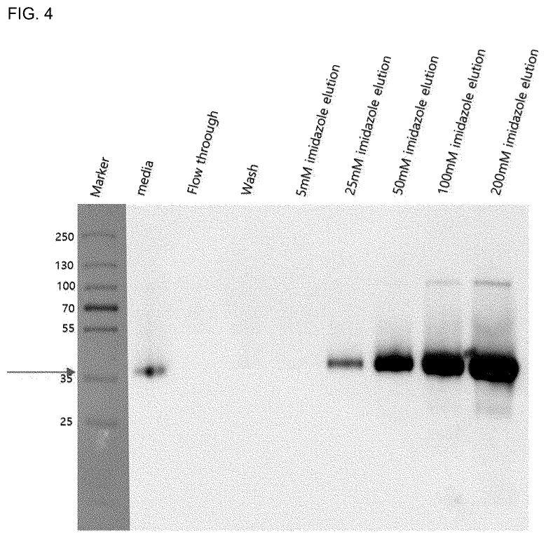

Specifically, SDS-PAGE was performed using 12% SDS-PAGE gel, and the CtsZ protein was transferred for 2 hours to a PVDF membrane which was activated with methanol for 10 minutes. The PVDF membrane to which the protein was transferred was blocked with PBST (0.05% Tween-20) containing 5% skim milk at room temperature for 2 hours, and then washed 4 times with 0.05% PBST, for 5 minutes each. After reacting with HRP-conjugated anti-6x His tag antibody diluted to 5000:1 at room temperature for 1 hour, washing was performed under the same condition. After chemiluminescence reaction for visualization of immunoreactive protein bands, the bands were detected with western blot LAS4000 (FIG. 4).

Referring to FIG. 4, it can be seen that, whereas the size of CtsZ predicted from the amino acid sequence was about 33 kDa, the actual size of CtsZ analyzed by SDS-PAGE was about 37 kDa. This may be due to the two glycosylation sites existing in the CtsZ gene (yield: 5.75 mg CtsZ/30 mL medium).

### 1-1-3. Measurement of Binding Affinity of Integrin αVβ3 Binding to CtsZ

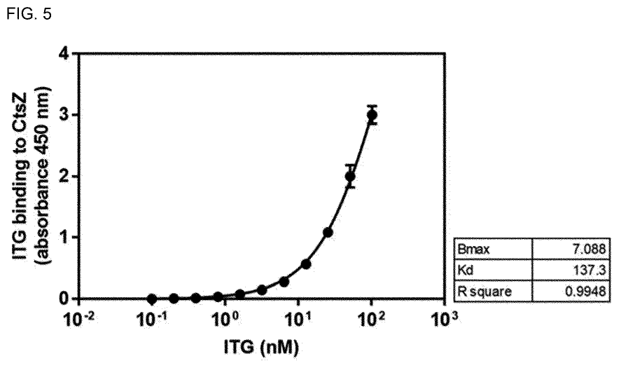

Because CtsZ has an RGD motif binding to integrin, particularly integrin αVβ3, in the pro-region, it was investigated whether the purified CtsZ was in active form by measuring binding affinity for integrin αVβ3 purchased from ACROBiosystems. As a result, CtsZ and integrin αVβ3 showed specific binding interaction and the K_{D} value was about 137 nM (FIG. 5).

### 1-2. Fixation of CtsZ-CNBr Resin and Biopanning of Single-Chain Surface Display Phage Library

Resin-antigen binding which maximizes surface exposure of antigens was selected instead of plate-antigen binding with less surface exposure of antigens. Whereas the antigen-binding ability is about 500 ng antigen/cm2 in the existing method, it is estimated that the antigen-binding ability increases by about 100 times to about 50 μg antigen/10 μL in the new method. Therefore, the phage display biopanning of the present disclosure provides higher possibility of finding the desired clone. Actually, in the resin-antigen binding test, the resin capacity was found to be about 56 μg CtsZ/10 μL. The resin-antigen binding test was performed using the nanodrop method ((amount of bound antigens)−(amount of bound antigens in buffer filtered by centrifuge filter tube after binding)). This method was performed using a centrifuge filter tube instead of a plate, and it is favorable in terms of panning cycles.

### 1-2-1. Fixation of CtsZ-CNBr Resin

1) Activation of CNBr resin: CNBr resin was washed with 1 mM HCl to remove impurities (additives). Then, the remaining HCl was washed with PBS for binding to CtsZ.

2) Binding of CtsZ and CNBr resin: The CNBr resin washed with PBS and CtsZ were mixed at a ratio of CNBr resin:CtsZ=100 μL:0.3 mg and incubated at 4° C. for 16 hours.

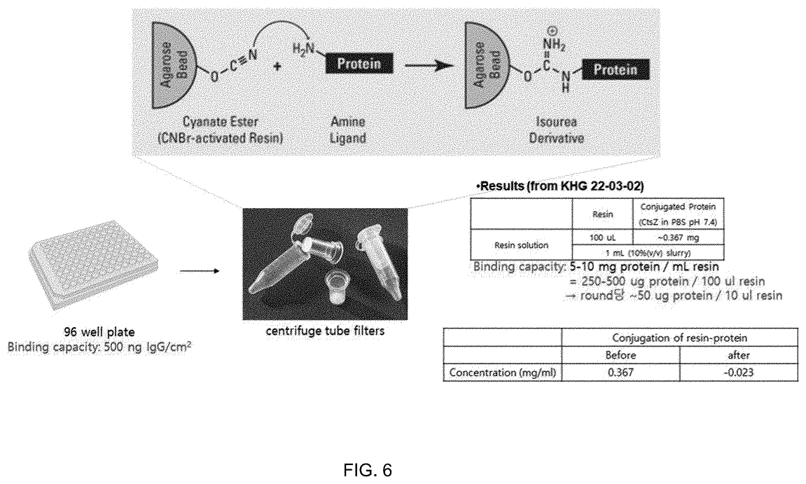

3) Blocking and washing: After washing with PBS using a spin column, incubation was performed at room temperature for 2 hours with 0.1 M Tris-Cl (pH 8.0) to block the active groups remaining in the CNBr resin. Then, after washing again with PBS, the CtsZ-bound CNBr resin was suspended in 0.9 mL of PBS and stored as a 10% (v/v) slurry at 4° C. (FIG. 6).

### 1-2-2. Biopanning of Single-Chain Surface Display Phage Library

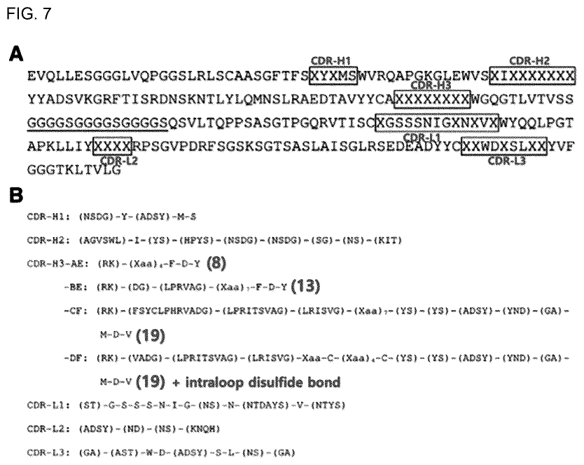

1) Phage binding: After placing 100 μL of the CtsZ-CNBr resin in a spin column and removing the buffer by centrifuging at 500 g for 10 seconds, 3% BSA was added and blocking was performed at room temperature for 2 hours. Then, after washing with PBS and adding the OPAL phage library (~2×10¹² CFU/200 μL), incubation was performed at 4° C. for 16 hours (FIG. 7) (Yang, H. Y. et al., (2009). Construction of a large synthetic human scFv library with six diversified CDRs and high functional diversity. Molecules and Cells, 27, 225-235).

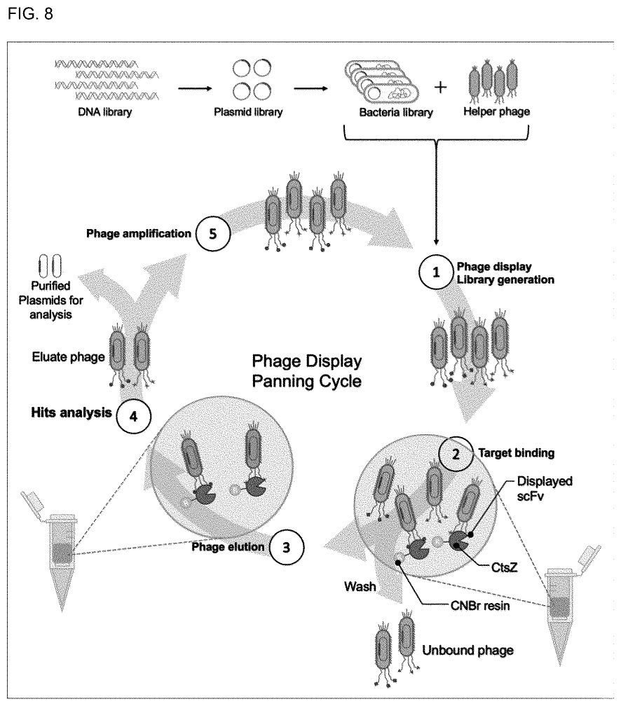

FIG. 8 schematically shows the process of biopanning according to the present disclosure.

2) Phage elution: After performing washing for each round of biopanning, a low-pH elution buffer (pH 2.2) was added and incubation was performed at room temperature for 10 minutes. Then, the phage eluate was stored at 4° C. after mixing well with a neutralization buffer (pH 8.0).

3) Phage amplification: After infecting ER2738 cells grown to OD₆₀₀=0.6 with the phage eluate, the cells were scaled up to 42 mL while incubating at 37° C. for 2 hours. After adding a helper phage M13KO7 (2.4×10¹² PFU/mL), incubation was performed at 37° C. for 1 hour. Then, after adding the kanamycin antibiotic, incubation was performed at 30° C. for 16 hours. After separating the cells by centrifuging at 3500 g for 30 minutes, 4% PEG and 3% NaCl were dissolved well in the supernatant and incubation was performed at 4° C. for 4 hours. Phage pellets obtained by centrifuging at 15000 g for 1 hour were resuspended in a 1% BSA/PBS buffer, filtered through a 0.2 μm-filter, and then stored at 4° C.

4) 1) to 3) were repeated until the 3 round biopanning, and the 4 round and 5 round biopanning were performed by omitting the amplification process of 3).

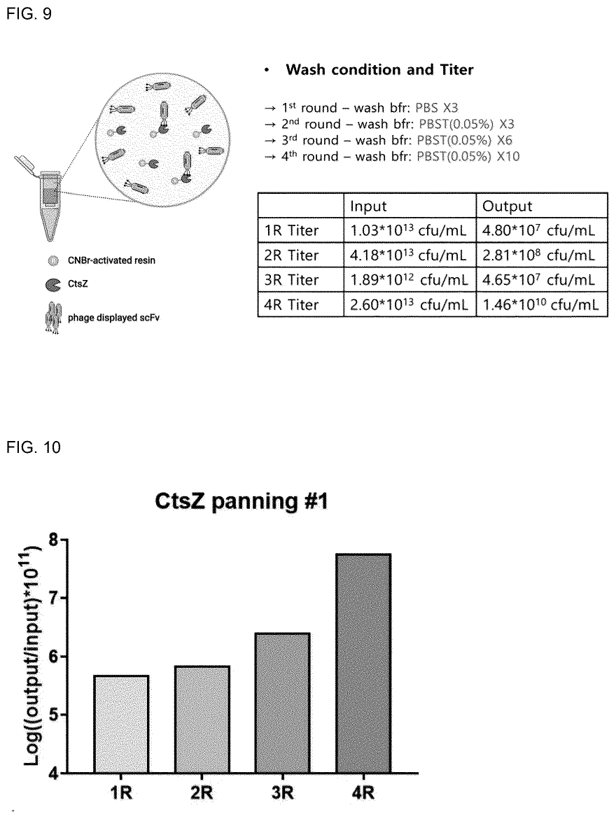

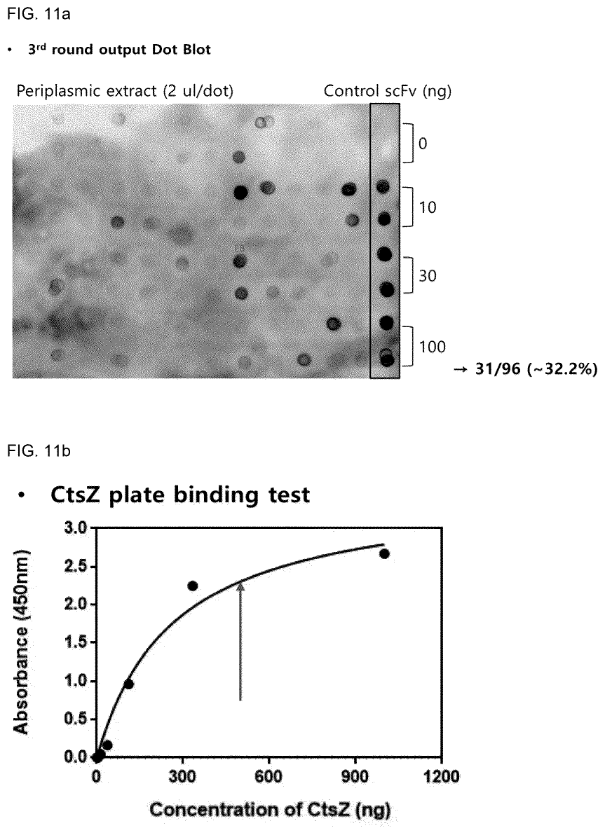

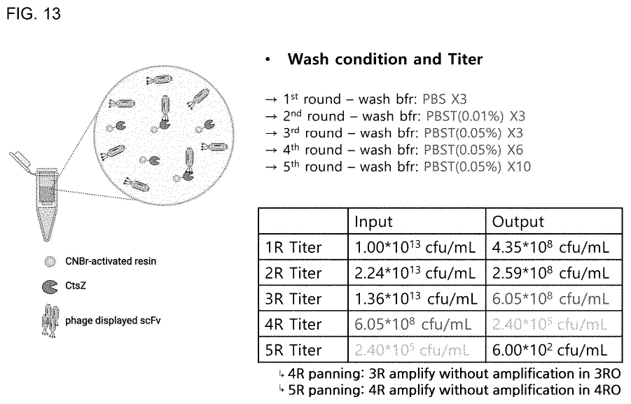

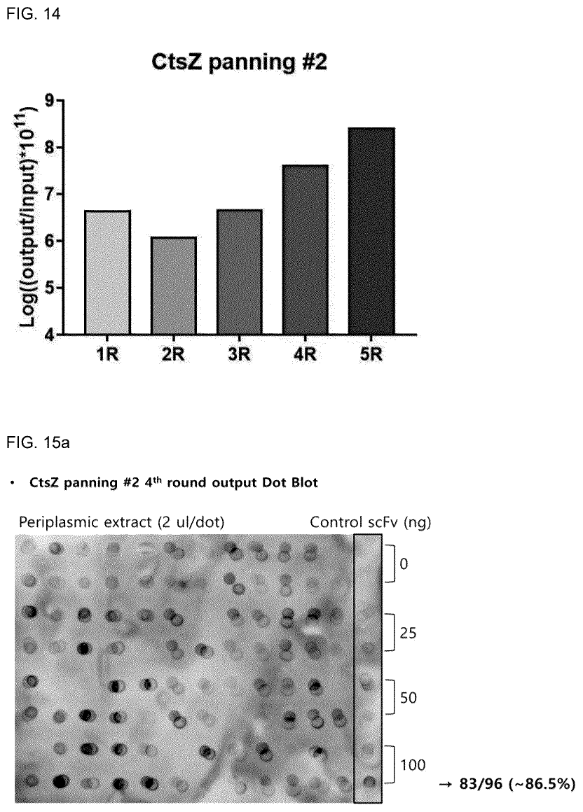

5) The ER2788 cells infected with the phage were plated on an LB/agar plate containing carbenicillin, and input and output phage titers were determined (FIG. 9, FIG. 10, FIG. 13 and FIG. 14).

Referring to FIG. 9, FIG. 10, FIG. 13 and FIG. 14, the titers increased in both the first biopanning (CtsZ biopanning #1) and second biopanning (CtsZ biopanning #2) with the pass of rounds, suggesting that scFv clones specific for CtsZ were enriched with the pass of biopanning rounds.

### 1-2-3. Dot Blot Assay

It was investigated the amplification process between the 3 round and the 4 round affect the change in the in-frame percentage of the biopanning output. Dot blot assay was performed to analyze the in-frame percentage of the biopanning output.

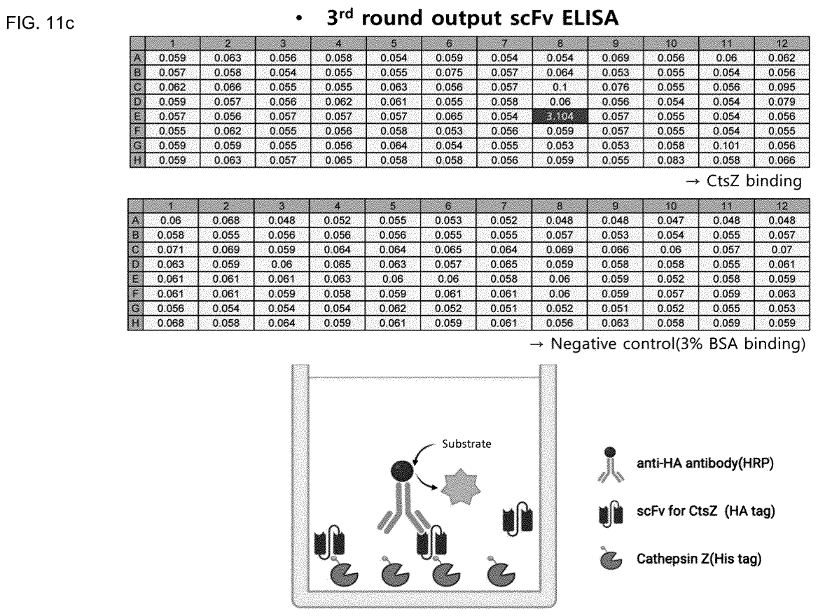

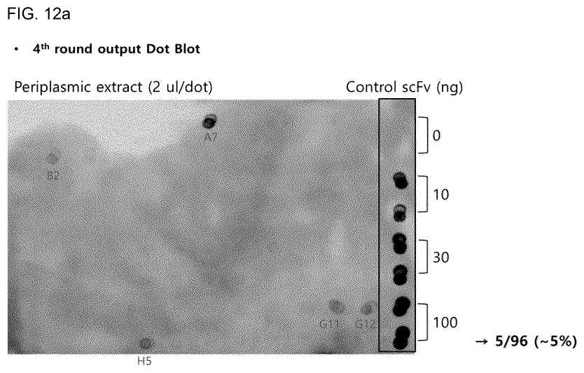

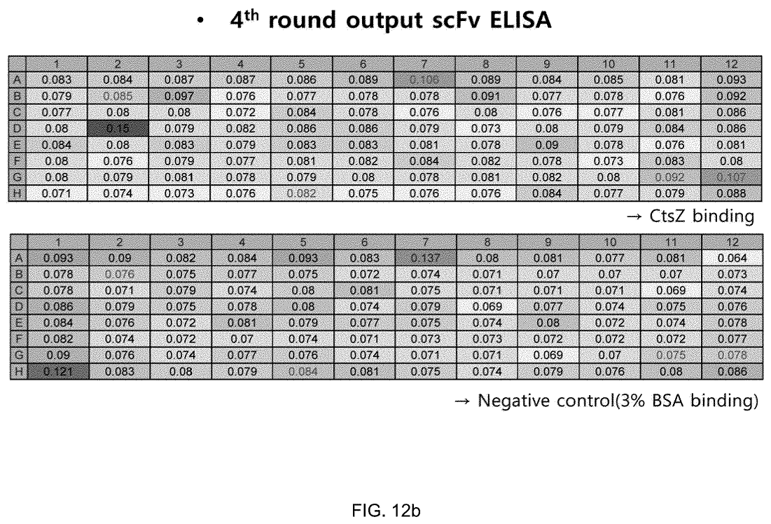

As a result of the first biopanning, when the amplification process was performed between the 3 round and the 4 round, the in-frame percentage of the 4 round output decreased rapidly as compared to the 3 round output (3 round output: 31/96, 32.2%→4 round output: 5/96, 5%) (FIG. 11a and FIG. 12a).

However, one CtsZ-specific clone was detected when scFv ELISA was conducted for the 3 round output (FIG. 11a and FIG. 11c). Therefore, in the second biopanning, the amplification process was omitted between the 3 round and the 4 round.

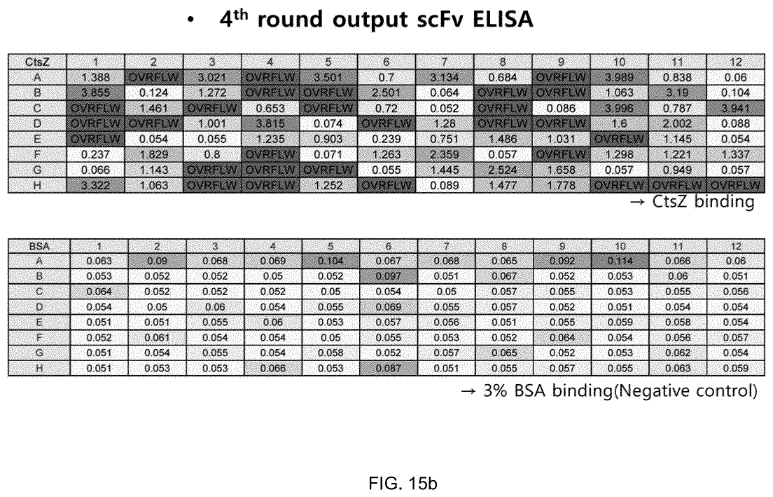

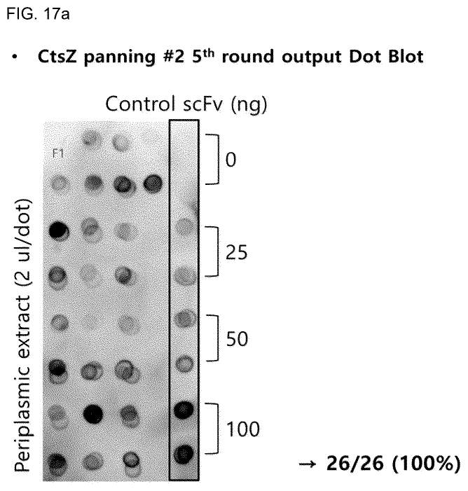

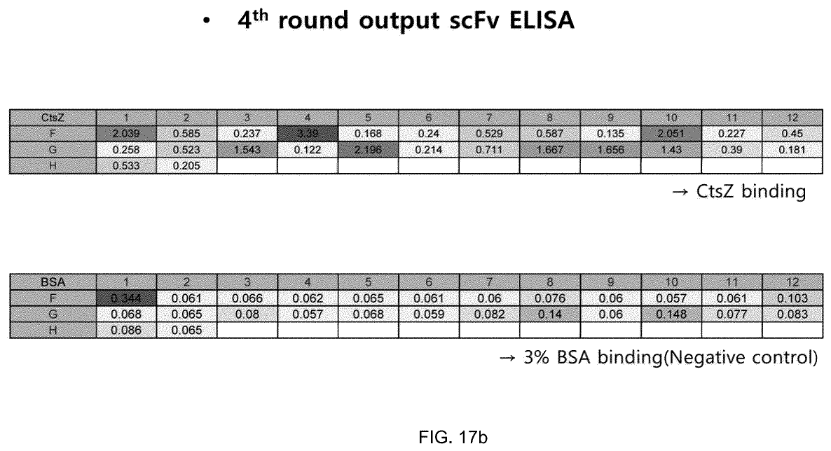

As a result of the second biopanning, when the 4 round and 5 round were performed without the amplification process, the in-frame percentage of the biopanning output did not decrease but increase on the contrary (4 round output: 83/96, 86%, 5 round output: 26/26, 100%) (FIG. 15a and FIG. 17a).

Meanwhile, plate binding test was conducted to investigate whether the purified CtsZ antigen binds to a plate (FIG. 11b). After binding CtsZ at different concentrations (1000 ng, ⅓ fold) onto a high-binding 96 well plate, the binding was detected using the HRP-conjugated anti-6X His antibody. As a result, it was confirmed that the purified CtsZ antigen was bound to the plate enough when the amount of the CtsZ bound was about 300 ng or more. Therefore, the subsequent ELISA experiment was conducted by fixing the amount of the CtsZ bound to the plate to 300 ng.

1-2-4. Screening of Single-Chain Antibody Clones Specific for CtsZ by scFv ELISA

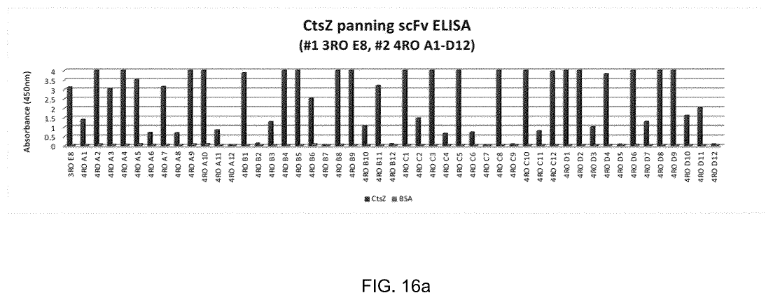

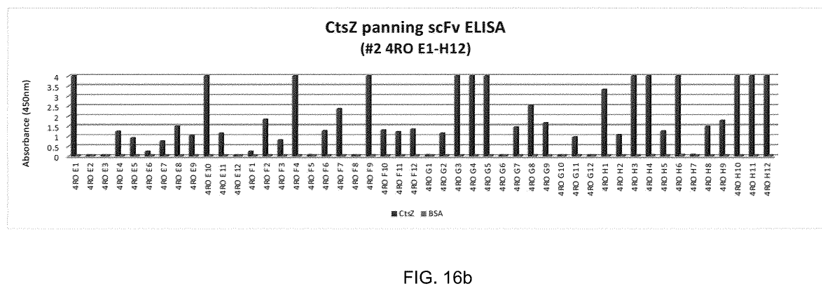

A total of 96 scFv clones specific for CtsZ were obtained by performing scFv ELISA and screening the clones with an intensity of 0.3 or higher (3 round output of first panning: 1, 4 round output of second panning: 76, 5 round output of second panning: 19) (FIG. 16a, FIG. 16b and FIG. 18).

### 1-2-5. Gene Sequencing of Selected Single-Chain Antibody Clones Specific for CtsZ

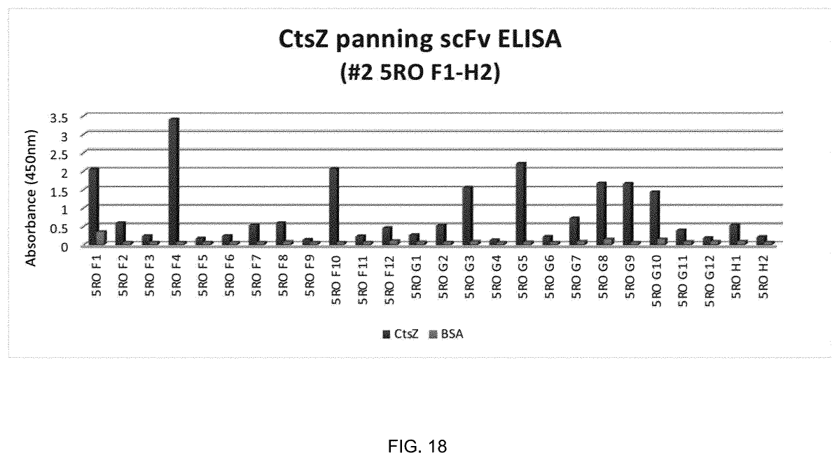

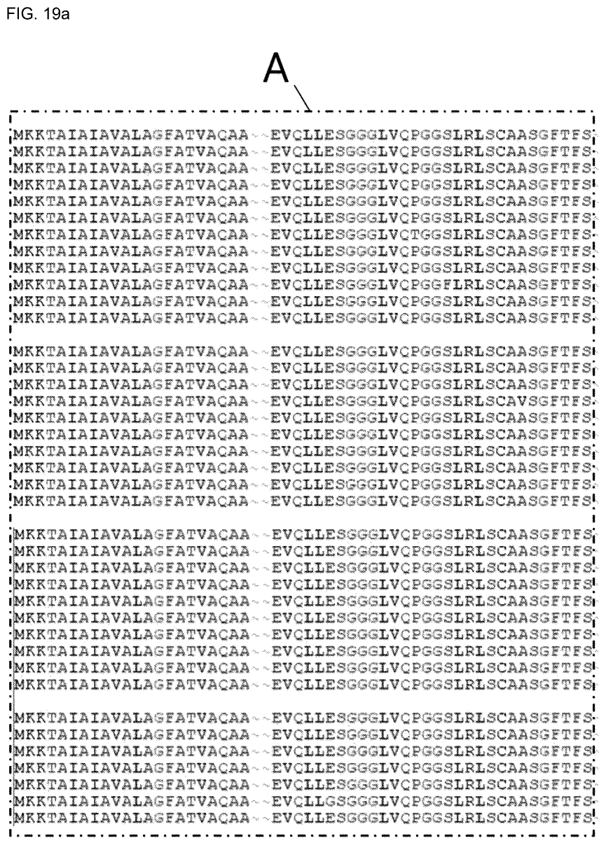

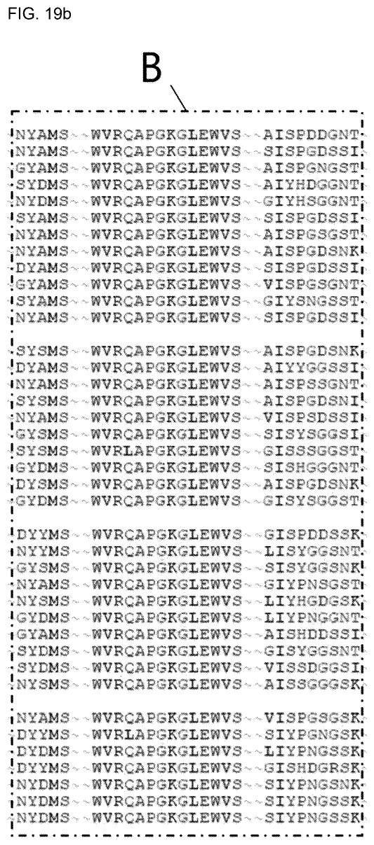

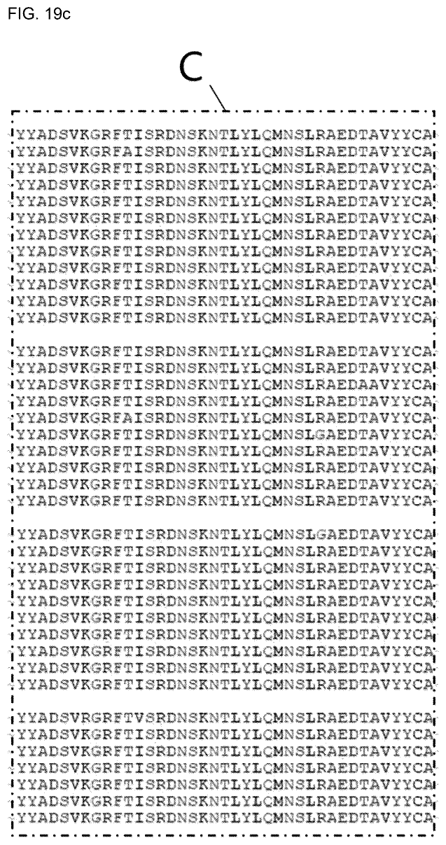

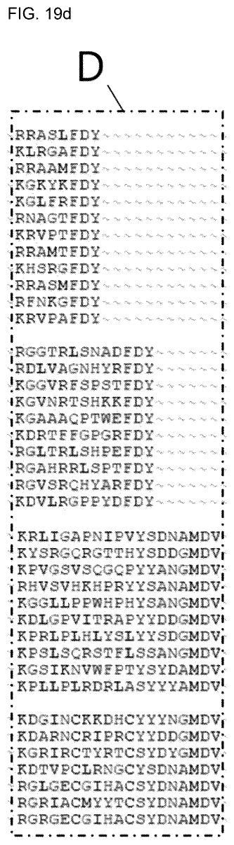

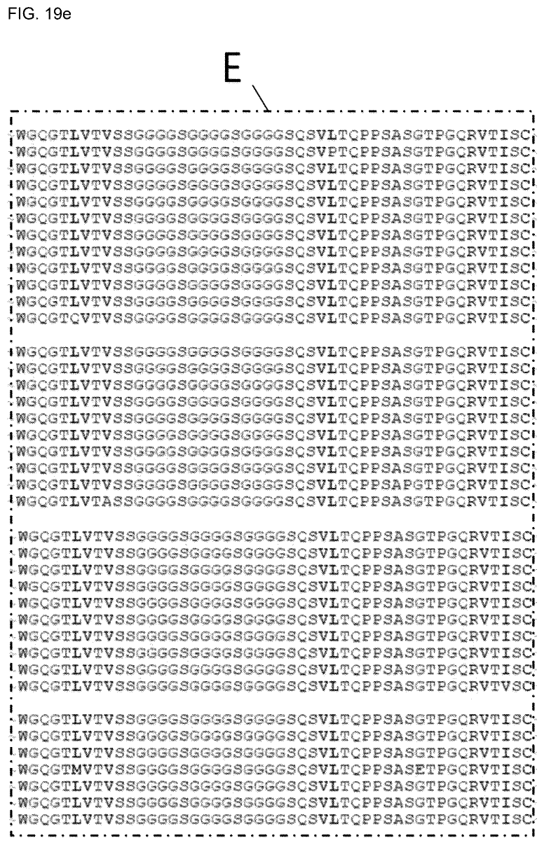

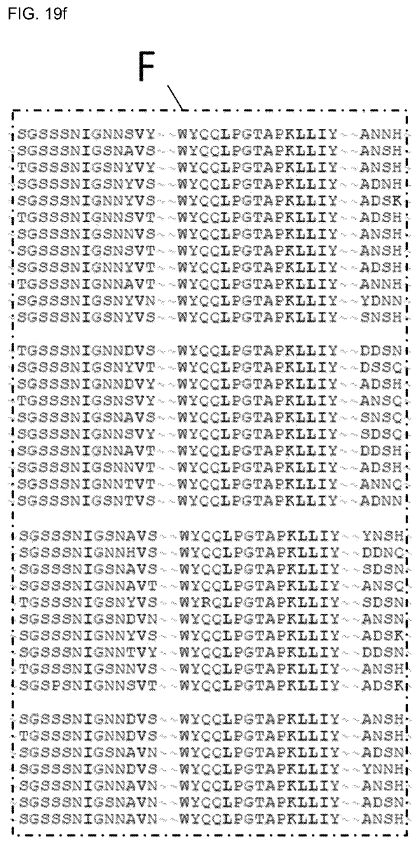

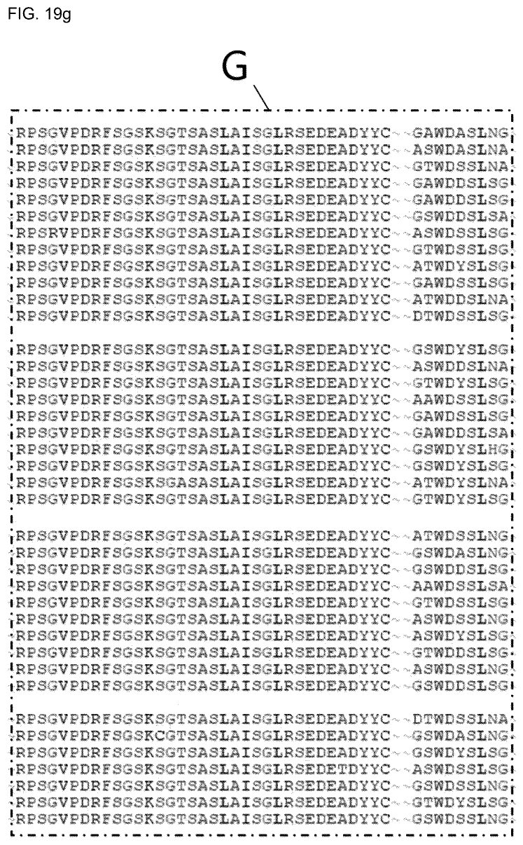

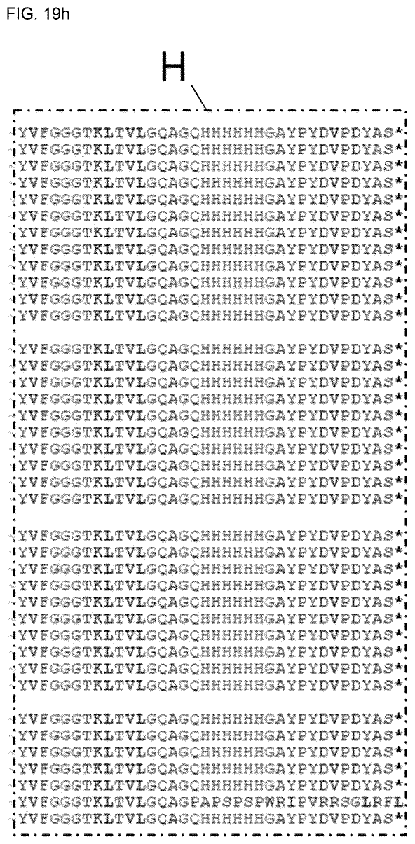

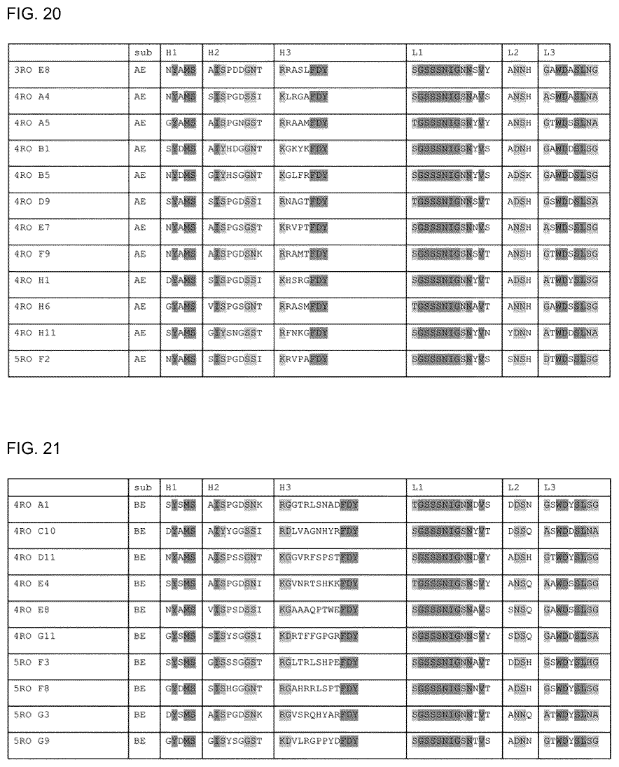

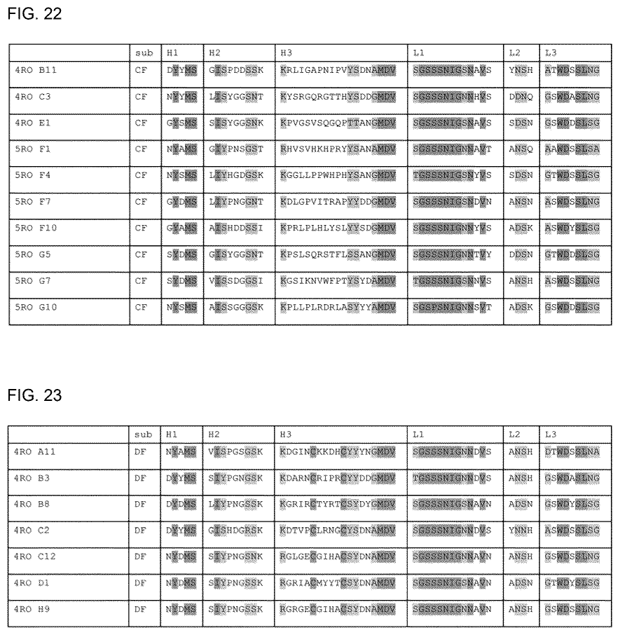

Through gene sequencing, a total of different 39 clones were obtained excluding enriched sequences (3 round output of first panning: 1, 4 round output of second panning: 30, 5 round output of second panning: 8) (FIG. 19 to FIG. 23).

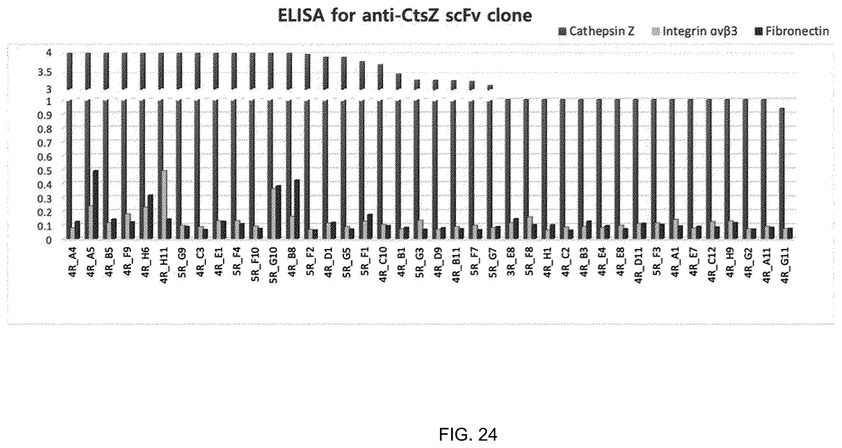

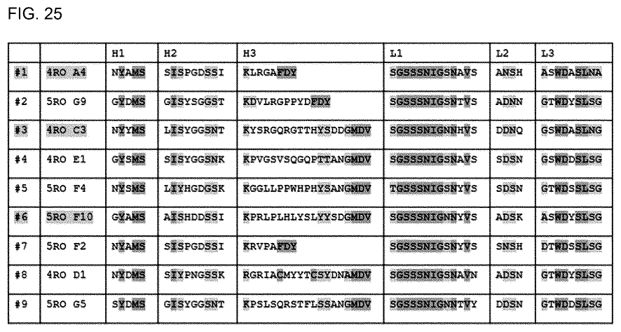

Then, in order to screen the scFv clones with stronger specificity for CtsZ from among the 39 different scFv clones, the scFv clones satisfying 1) specific binding for CtsZ with high intensity, 2) nonspecific binding for integrin αVβ3 with low intensity, and 3) nonspecific binding for fibronectin with low intensity were screened through ELISA. As a result, 9 CtsZ-specific scFv clones satisfying all of the 3 conditions were determined (#1: 4R_A4, #2: 5R_G9, #3: 4R_C3, #4: 4R_E1, #5: 5R_F4, #6: 5R_F10, #7: 5R_F2, #8: 4R_D1, #9: 5R_G5) (FIGS. 24 and 25).

1-3. Purification and Binding Affinity of CtsZ-Specific scFv

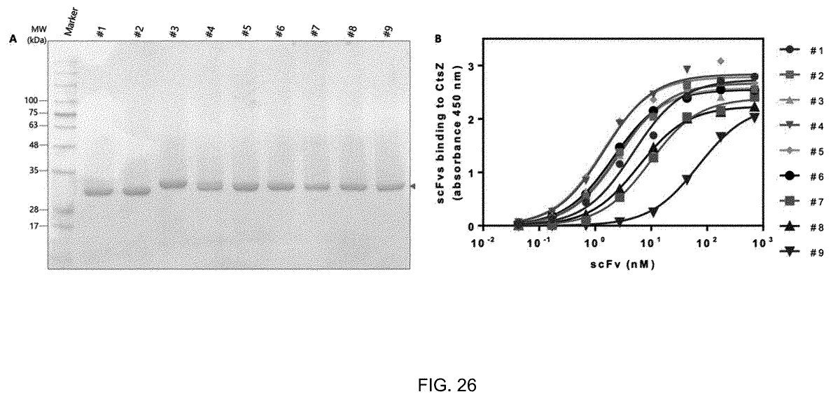

Because the screening of the CtsZ-specific scFv clones was performed using the scFv-expressing periplasmic fraction, it is necessary to examine the purified scFv clones individually for a more accurate result. Therefore, a periplasmic fraction was obtained for each clone by expressing in TOP10F cells by a pComb3X plasmid and applying osmotic shock, and was purified by Ni-NTA affinity chromatography. As a result of SDS-PAGE analysis, the screened 9 scFv clones showed purity of 95% or higher (FIG. 26). In addition, the K_{D} values of the scFv clones for CtsZ were determined by measuring binding affinity through ELISA. The K_{D} values of scFv clones #1, #2, #3, #4, #5, #6, #7, #8 and #9 for CtsZ in nanomolar units are as follows (K_{D} values, #1:5.9 nM, #2:3.1 nM, #3:3.0 nM, #4:1.5 nM, #5:1.7 nM, #6:2.7 nM, #7:11.4 nM, #8:6.8 nM, #9:69.1 nM) (FIG. 26).

### Test Example 2: Analysis of Effect of Treatment with Anti-CtsZ Antibody on Death of MES-Type Brain Tumor Stem Cells

The effect of the anti-CtsZ antibody of the present disclosure on the death of MES-type brain tumor stem cells was investigated by treating the anti-CtsZ antibody prepared in Test Example 1 to MES-type brain tumor stem cells.

Specifically, after treating the anti-CtsZ antibody prepared in Test Example 1 (#6) to MES-type brain tumor stem cells (83NS cells) (75 μg/mL), the subG1 fraction showing cell death was determined by analyzing the cell cycle using the FACS instrument (Mylteni, MACSQuant analyzer 10). The 83NS cells were cultured in a medium containing a B27 supplement (0.04% v/v) and EGF/bFGF (20/10 ng/ml) in DMEM/F12, and the concentration of the cells during antibody treatment was 5×10⁴/mL.

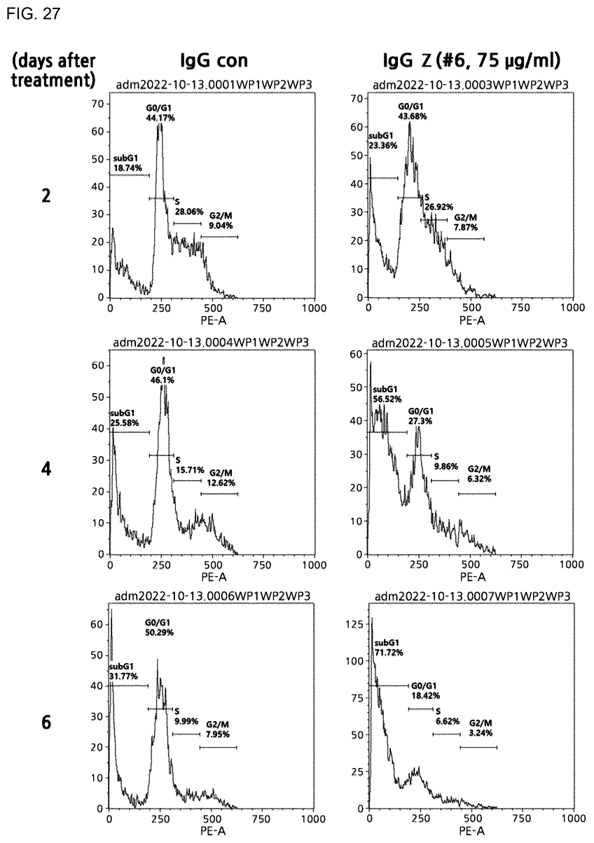

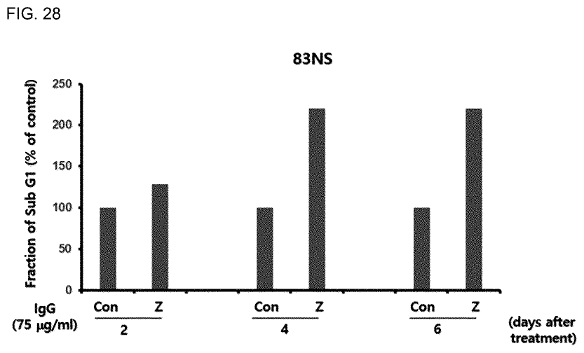

As a result, the subG1 fraction showing the death of the 83NS cells increased remarkably by treatment with the anti-CtsZ antibody of the present disclosure, suggesting that the anti-CtsZ antibody kills the MES brain tumor stem cells effectively (FIG. 27 and FIG. 28).

From the above description, it will be understood by those having ordinary knowledge in the art to which the present disclosure belongs that the present disclosure can be embodied in different specific forms without changing the technical idea or essential characteristics thereof. In this regard, it should be understood that the above-described exemplary embodiments are merely illustrative and not limitative in all aspects. The present disclosure is defined by the appended claims and encompasses all modifications and alterations derived from the meanings, scope and equivalents of the appended claims.

1. A monoclonal antibody comprising any heavy chain variable region and light chain variable region selected from a group consisting of 1) to 9) and specifically binding to cathepsin Z, or an antigen-binding fragment thereof:
1) a heavy chain variable region comprising a CDR1 region represented by SEQ ID NO: 1, a CDR2 region represented by SEQ ID NO: 2 and a CDR3 region represented by SEQ ID NO: 3 and a light chain variable region comprising a CDR1 region represented by SEQ ID NO: 4, a CDR2 region represented by SEQ ID NO: 5 and a CDR3 region represented by SEQ ID NO: 6;
2) a heavy chain variable region comprising a CDR1 region represented by SEQ ID NO: 7, a CDR2 region represented by SEQ ID NO: 8 and a CDR3 region represented by SEQ ID NO: 9, and a light chain variable region comprising a CDR1 region represented by SEQ ID NO: 10, a CDR2 region represented by SEQ ID NO: 11 and a CDR3 region represented by SEQ ID NO: 12;
3) a heavy chain variable region comprising a CDR1 region represented by SEQ ID NO: 13, a CDR2 region represented by SEQ ID NO: 14 and a CDR3 region represented by SEQ ID NO: 15, and a light chain variable region comprising a CDR1 region represented by SEQ ID NO: 16, a CDR2 region represented by SEQ ID NO: 17 and a CDR3 region represented by SEQ ID NO: 18;
4) a heavy chain variable region comprising a CDR1 region represented by SEQ ID NO: 19, a CDR2 region represented by SEQ ID NO: 20 and a CDR3 region represented by SEQ ID NO: 21, and a light chain variable region comprising a CDR1 region represented by SEQ ID NO: 22, a CDR2 region represented by SEQ ID NO: 23 and a CDR3 region represented by SEQ ID NO: 24;
5) a heavy chain variable region comprising a CDR1 region represented by SEQ ID NO: 25, a CDR2 region represented by SEQ ID NO: 26 and a CDR3 region represented by SEQ ID NO: 27, and a light chain variable region comprising a CDR1 region represented by SEQ ID NO: 28, a CDR2 region represented by SEQ ID NO: 29 and a CDR3 region represented by SEQ ID NO: 30;
6) a heavy chain variable region comprising a CDR1 region represented by SEQ ID NO: 31, a CDR2 region represented by SEQ ID NO: 32 and a CDR3 region represented by SEQ ID NO: 33, and a light chain variable region comprising a CDR1 region represented by SEQ ID NO: 34, a CDR2 region represented by SEQ ID NO: 35 and a CDR3 region represented by SEQ ID NO: 36;
7) a heavy chain variable region comprising a CDR1 region represented by SEQ ID NO: 37, a CDR2 region represented by SEQ ID NO: 38 and a CDR3 region represented by SEQ ID NO: 39, and a light chain variable region comprising a CDR1 region represented by SEQ ID NO: 40, a CDR2 region represented by SEQ ID NO: 41 and a CDR3 region represented by SEQ ID NO: 42;
8) a heavy chain variable region comprising a CDR1 region represented by SEQ ID NO: 43, a CDR2 region represented by SEQ ID NO: 44 and a CDR3 region represented by SEQ ID NO: 45, and a light chain variable region comprising a CDR1 region represented by SEQ ID NO: 46, a CDR2 region represented by SEQ ID NO: 47 and a CDR3 region represented by SEQ ID NO: 48; and
9) a heavy chain variable region comprising a CDR1 region represented by SEQ ID NO: 49, a CDR2 region represented by SEQ ID NO: 50 and a CDR3 region represented by SEQ ID NO: 51, and a light chain variable region comprising a CDR1 region represented by SEQ ID NO: 52, a CDR2 region represented by SEQ ID NO: 53 and a CDR3 region represented by SEQ ID NO: 54.

2. The monoclonal antibody or an antigen-binding fragment thereof according to claim 1, wherein the antigen-binding fragment is selected from a group consisting of Fab, Fab′, F(ab′)₂, scFv, Fv, dsFv, a diabody, Fd and Fd′.

3. A polynucleotide encoding monoclonal antibody or an antigen-binding fragment thereof according to claim 1.

4. The polynucleotide according to claim 3, wherein the polynucleotide is represented by any sequence selected from SEQ ID NO: 73 to SEQ ID NO: 81.

5. An expression vector comprising the polynucleotide according to claim 3.

6. A transgenic organism transfected with the expression vector according to claim 5.

7. A method for preventing or treating cancer in which cathepsin Z is overexpressed, comprising a step of administering the monoclonal antibody or an antigen-binding fragment thereof according to claim 1, a polynucleotide encoding the monoclonal antibody or an antigen-binding fragment thereof, or an expression vector comprising the polynucleotide to a subject in need thereof.

8. The method according to claim 7, wherein the cancer is one or more selected from brain cancer, liver cancer, colorectal cancer and prostate cancer.

9. The method according to claim 8, wherein the brain cancer is malignant brain cancer or brain tumor.

10. The method according to claim 9, wherein the malignant brain cancer is glioblastoma or glioblastoma multiforme.

11. The method according to claim 9, wherein the brain tumor is anaplastic astrocytoma.

12. A composition for diagnosing cancer in which cathepsin Z is overexpressed, comprising the monoclonal antibody or an antigen-binding fragment thereof according to claim 1, a polynucleotide encoding the monoclonal antibody or an antigen-binding fragment thereof, or an expression vector comprising the polynucleotide.

13. A kit for diagnosing cancer in which cathepsin Z is overexpressed, comprising the composition according to claim 12 and an instruction.

14. A method for providing information necessary for diagnosis of cancer in which cathepsin Z is overexpressed, comprising:
(a) a step of contacting the monoclonal antibody or an antigen-binding fragment thereof according to claim 1, a polynucleotide encoding the monoclonal antibody or an antigen-binding fragment thereof, or an expression vector comprising the polynucleotide with a biological sample isolated from a subject suspected of cancer in which cathepsin Z is overexpressed;
(b) a step of measuring the expression level of the cathepsin Z protein in the biological sample; and
(c) a step of diagnosing as cancer in which cathepsin Z is overexpressed if the expression level of the cathepsin Z protein measured in the step (b) is higher as compared to a control group.

15. (canceled)
